(19) United States
(12) Patent Application Publication
(10) Pub. No.: US 2009/0311203 A1
(43) Pub. Date: Dec. 17, 2009

(54) GEM-DIFLUORIDE C-GLYCOPEPTIDE COMPOUNDS, THEIR PREPARATION AND USE PARTICULARLY FOR PRESERVATION OF BIOLOGICAL MATERIALS

(75) Inventors: Géraldine Castelot Deliencourt-Godefroy, Rouen (FR); Jean-Charles Quirion, Bourg-Achard (FR)

Correspondence Address: BROWDY AND NEIMARK, P.L.L.C.;624 NINTH STREET, NW, SUITE 300, WASHINGTON, DC, 20001-5303, US

(73) Assignee: INSTITUT NATIONAL DES SCIENCES APPLIQUEES DE ROUEN (INSA), Mont Saint Aignan Cedex (FR)

(21) Appl. No.: 12/299,453

(22) PCT Filed: Apr. 26, 2007
(86) PCT No.: PCT/FR2007/000716

§ 371 Date: Feb. 25, 2009

(30) Foreign Application Priority Data
May 3, 2006 (FR) 0603952

(51) Int. Cl. A61K 8/18; C07K 2/00; A61K 38/16; A61Q 17/04; C12N 5/02

(52) U.S. Cl. 424/59; 530/300; 514/8; 435/374

(57) ABSTRACT

The invention relates to a gem-difluoride C-glycopeptide compound with formula I where n is an integer equal to 3 or 4, R represents a hydrogen atom, an alkyl, benzyl, acetyl, trimethylsilyl, tert-butyldimethylsilyl, tert-butyldiphenylsilyl group, R′ represents OR, NR′R″, N₃, or a phthalimide, R′ and R″ represent a hydrogen atom or an alkyl, aryl, benzyl, benzoyl, acetyl, alkyloxycarbonyl, allyloxycarbonyl, benzyloxycarbonyl group, R1 represents a hydrogen atom or an alkyl, benzyl, alkylcarbamate, allylcarbamate, benzylcarbamate, acetyl group, R₁ may consist of an amino acid if R₂ represents only OR, R₂ includes an amino acid if R₁ represents a hydrogen atom or an alkyl, benzyl, alkylcarbamate, allylcarbamate, benzylcarbamate, acetyl group, R₂ represents OR when R₁ represents an amino acid, R₃ represents a hydrogen atom or a free or protected alcohol function. It is applicable to the preparation of compounds or compositions to be used for preservation of biological materials such as cells, tissues and organs at various temperatures.

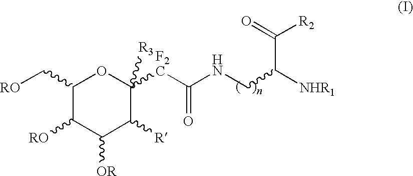

The invention relates to a method for the synthesis of gem-difluorinated C-glycopeptide compounds. It is more specifically, but not exclusively, applicable to the preparation of compounds or formulations that can be used particularly for the preservation of biological materials such as cells, tissues and organs at different temperatures, but also in the treatment of inflammation.

A family of anti-freeze glycoproteins present particularly in some fish and enabling them to survive in an environment where the temperatures are approximately or less than 0° C., has displayed a biological material protection activity in numerous studies at low or very low temperatures (approximately +4° C. to −196° C.).

For many years, scientists have been studying the influence of these anti-freeze compounds extracted from the natural environment (fish, amphibians, plants, insects, etc.) and are moving towards the synthesis of sufficiently stable analogue compounds and displaying an at least equal or superior activity to natural molecules for commercial applications.

In fact, these natural compounds display numerous limitations:

difficult extraction from the natural environment or synthesis,

complex analyses with low purities,

unstable products with respect to chemical and enzyme hydrolyses.

Glycoproteins, due to the presence of an oside bond (bond involving oxygen said to be in an anomeric position), are fragile with respect to several enzyme systems including glycosidase enzymes and are also sensitive to acid-base hydrolysis, which makes the synthesis thereof more difficult.

Therefore, it is beneficial to enable compounds to preserve their biological properties, to replace the oxygen in the oside bond so that the bond is no longer degraded by an enzyme process.

Analogues where oxygen is replaced by a CH₂ group have been synthesised, but, in spite of an increase in stability and a steric size similar to that of oxygen, the CH₂ group has not always proved to be a good mimic of osidic oxygen. As a result, the biological properties of the initial compound are not necessarily obtained.

Other classes of compounds where oxygen is replaced by a nitrogen or a sulphur and more recently by a difluoromethylene group are studied in order to give the glycoconjugate compounds increased stability in a biological medium.

In fact, the CF₂ group is particularly resistant to biochemical degradation processes and, therefore, it enables the synthesis of non-hydrolysable structures.

This O/CF₂ transposition seems to be particularly suitable for mimicking oxygen in terms of electrons; both fluorine atoms playing the role of both free oxygen doublets.

The Applicant has developed gem-difluoroglycopeptides which have demonstrated a very strong preservation activity of various cell lines at temperatures varying from −196° C. to +37° C.

In fact, structural modifications made with respect to the native compounds and the observation of a genuine anti-apoptosis effect, i.e. no cell lysis, at physiological temperatures led the Applicant to extend the spectrum of activity of the compounds.

These compounds were named AAGP for Anti Aging GlycoProteins.

With a view to developing new analogues which are even more active, i.e. providing superior preservation of biological materials and with, in the first instance, the aim to improve the preservation of fibroblasts for applications in cosmetics, we looked at the synthesis of lower molecular weight compounds thus potentially having a superior bioavailability while simplifying the synthesis steps.

Such compounds would be usable with a view to numerous applications such as the preservation of cells, blood platelets, tissues and organs.

In fact, there is a strong demand to improve the storage and preservation of live materials with much less damage than with the methods generally used.

The term preservation is more generally used, including preservation at different temperatures including cryopreservation up to temperatures of −196° C.

In this way, compounds used as adjuvants during preservation and displaying good stability could be beneficial for preserving biological materials, particularly in the medical field:

to keep whole human organs such as kidneys, hearts and livers to be transplanted without being subject to time constraints,

to preserve cells or delicate tissue with minimum damage and for enough time to enable the potentially international distribution thereof,

to preserve blood platelets and various cells,

to protect some organisms, bacteria, viruses or vaccines,

but also in the field of dermatology and/or cosmetics to protect skin against damage caused by oxidative stress or UV or by aging, for example.

The aim of the invention is to resolve these drawbacks.

For this purpose, it proposes a gem-difluorinated C-glycopeptide compound according to formula I:

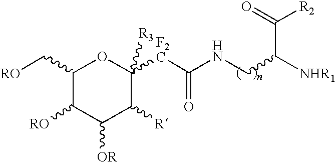

where n is an integer between 3 and 4,

R represents a hydrogen atom, a linear or branched alkyl, benzyl, acetyl, trimethylsilyl, tert-butyldimethylsilyl, tert-butyldiphenylsilyl group,

R′ represents OR, NR″R′″, N₃, or a phthalimide,

R″ and R′″, identical or different, represent a hydrogen atom or a linear or branched alkyl, aryl, benzyl, benzoyl, acetyl, alkyloxycarbonyl, allyloxycarbonyl, benzyloxycarbonyl group,

R₁ represents a hydrogen atom or a linear or branched alkyl, benzyl, alkylcarbamate, allylcarbamate, benzylcarbamate, acetyl group, R₁ may also represent an amino acid, but in this case R₂ only represents OR,

R₂ represents an amino acid, but in this case R₁ represents a hydrogen atom or a linear or branched alkyl, benzyl, alkylcarbamate, allylcarbamate, benzylcarbamate, acetyl group, R₂ represents OR when R₁ represents an amino acid,

R₃ represents a hydrogen atom or a free or protected alcohol function,

and the derivatives thereof in the form of a physiologically or pharmaceutically acceptable base, addition salt to a mineral or organic acid, hydrate or solvate.

The linear or branched alkyl groups may be groups comprising 1 to 10 carbon atoms.

Said amino acid may be an alanine or a glycine or a proline.

The term physiologically acceptable signifies compatible with skin, lips, scalp and/or hair.

The invention also relates to a medicinal product comprising as the active ingredient at least one gem-difluorinated C-glycopeptide according to formula I as defined above.

According to another of the aspects thereof, the present invention relates to the use of at least one gem-difluorinated C-glycopeptide compound according to formula I for the preparation of medicinal products intended to treat inflammation.

The invention also relates to the use of a gem-difluorinated C-glycopeptide compound according to formula I for the preparation of compounds or formulations that can be used for the preservation or cryopreservation of biological materials such as fibroblasts.

The invention also relates to a formulation comprising at least one gem-difluorinated C-glycopeptide compound according to formula I as defined above.

Naturally, the formulation according to the invention may comprise a gem-difluorinated C-glycopeptide compound according to formula I alone or in a mixture and in any proportions.

The formulation according to the invention may be intended for a cosmetic or pharmaceutical or particularly dermatological use.

The formulation may be ingested, injected or applied on the skin, lips, scalp and/or hair.

Depending on the mode of administration, the formulation according to the invention may be presented in any of the pharmaceutical forms normally used.

The formulation may comprise a physiologically or pharmaceutically acceptable medium and/or substrate.

In the pharmaceutical formulations according to the present invention for oral, sublingual, inhaled, subcutaneous, intramuscular, intravenous, transdermal, local or rectal administration, the active ingredients may be administered in unitary administration forms, mixed with conventional pharmaceutically acceptable substrates.

The unitary administration forms comprise oral forms such as tablets, capsules, powders, granules and solutions or oral suspensions, topical administration forms, implants, subcutaneous, intramuscular, intravenous, intranasal or intra-ocular administration forms and rectal administration forms.

In addition to inert, non-toxic and pharmaceutically acceptable excipients, such as distilled water, glucose, starch lactose, talc, vegetable oils, ethylene glycol, etc., the formulations obtained in this way may also contain preservation agents.

Other active ingredients may be added to these formulations.

The quantity of compound according to the invention and any other active ingredients in such formulations may vary according to the applications, the age and weight of the patient or the user, if applicable.

For a topical application on the skin, lips, scalp and/or hair, the formulation according to the invention may comprise a physiologically acceptable medium and/or substrate.

In addition, the formulation may be presented in any of the pharmaceutical forms normally used for a topical application, particularly in the form of an aqueous, hydroalcoholic or oily solution, an oil-in-water or multiple emulsion, an aqueous or oily gel, a liquid, pasty or solid anhydrous product, an oil dispersion in an aqueous phase using spherules that may be micro/nanocapsules or micro/nanoparticles, ionic and/or non-ionic vesicular dispersions.

This formulation may be more or less fluid and have the appearance of a white or coloured cream, ointment, milk, lotion, serum, paste, foam.

It may also be in solid form and, for example, in stick form.

It may be used as a personal care product, as a cleansing product, as a make-up product.

The formulation according to the invention may also be a hair care formulation, particularly a shampoo, a conditioning lotion, a cream or a styling gel.

The formulation may also contain the usual adjuvants in the cosmetic or dermatological fields.

The quantities of the various adjuvants are those conventionally used in the fields in question.

These adjuvants, depending on their nature, may be introduced in an aqueous phase, an oily phase, in vesicles and/or in micro/nanoparticles.

It is understood that these adjuvants and the concentration thereof should be such that they do not modify the desired property for the formulation according to the invention.

The invention also relates to a cosmetic treatment method to protect the skin, lips and/or hair, scalp against oxidative stress and/or UV consisting of applying on the skin, lips and/or hair, scalp, a formulation comprising at least one physiologically acceptable medium and at least one gem-difluorinated C-glycopeptide compound according to formula I as defined above or any of the derivatives thereof in the form of a physiologically or pharmaceutically acceptable base, addition salt to a mineral or organic acid, hydrate or solvate.

Embodiments of the invention will be described below, as non-limitative examples, with reference to the appended figures wherein:

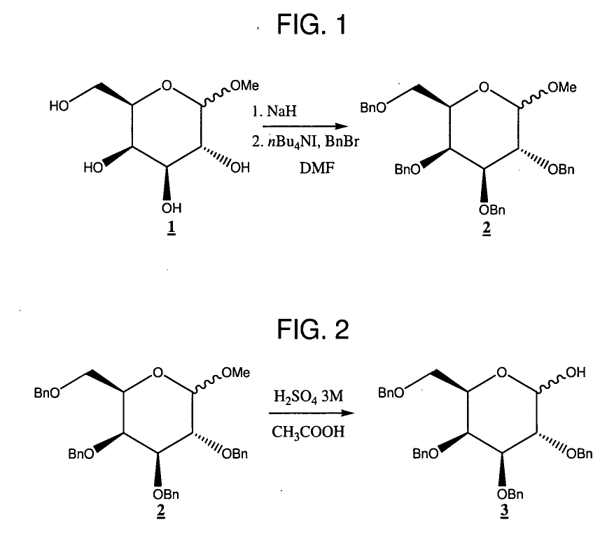

FIG. 1 is a reaction equation to obtain compound 2;

FIG. 2 is a reaction equation to obtain compound 3;

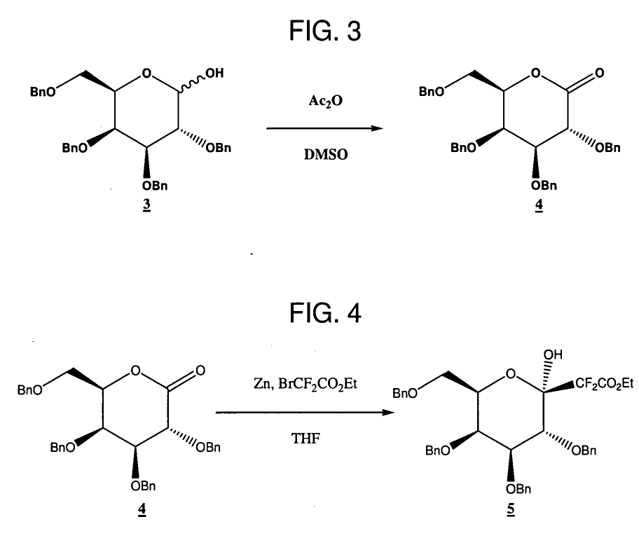

FIG. 3 is a reaction equation to obtain compound 4;

FIG. 4 is a reaction equation to obtain compound 5;

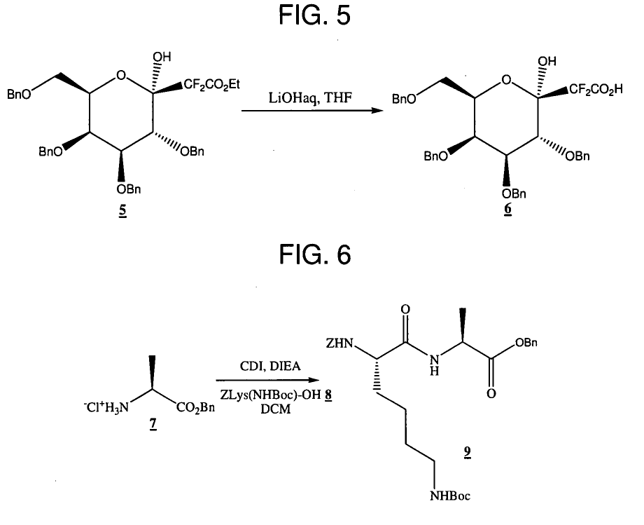

FIG. 5 is a reaction equation to obtain compound 6;

FIG. 6 is a reaction equation to obtain compound 9;

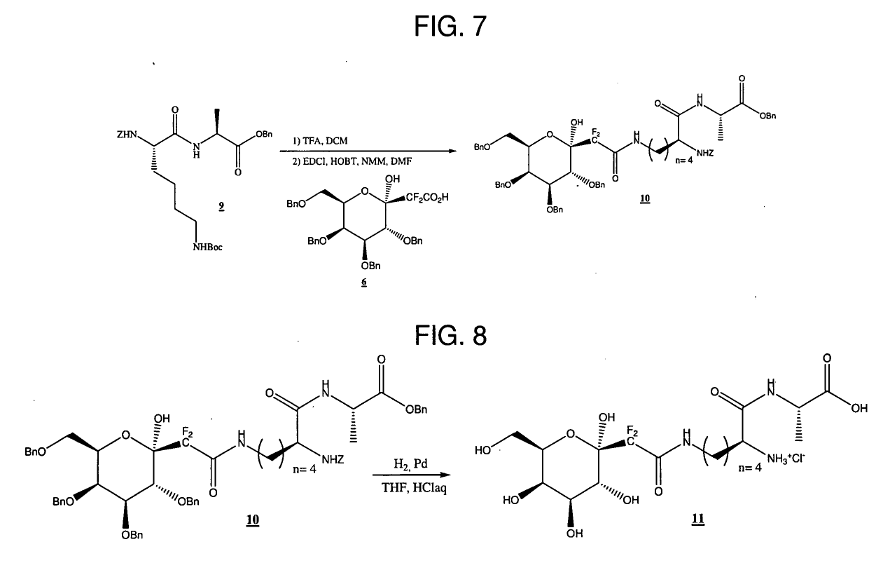

FIG. 7 is a reaction equation to obtain compound 10;

FIG. 8 is a reaction equation to obtain compound 11;

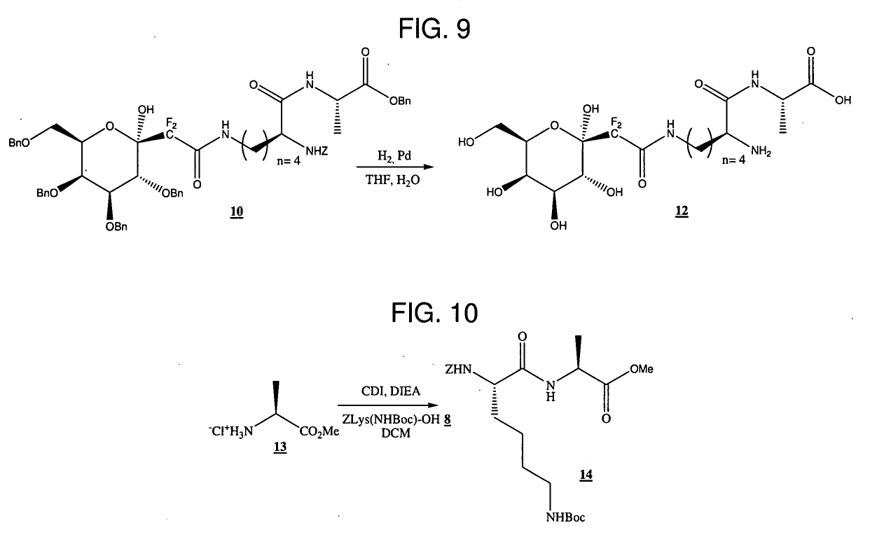

FIG. 9 is a reaction equation to obtain compound 12;

FIG. 10 is a reaction equation to obtain compound 14;

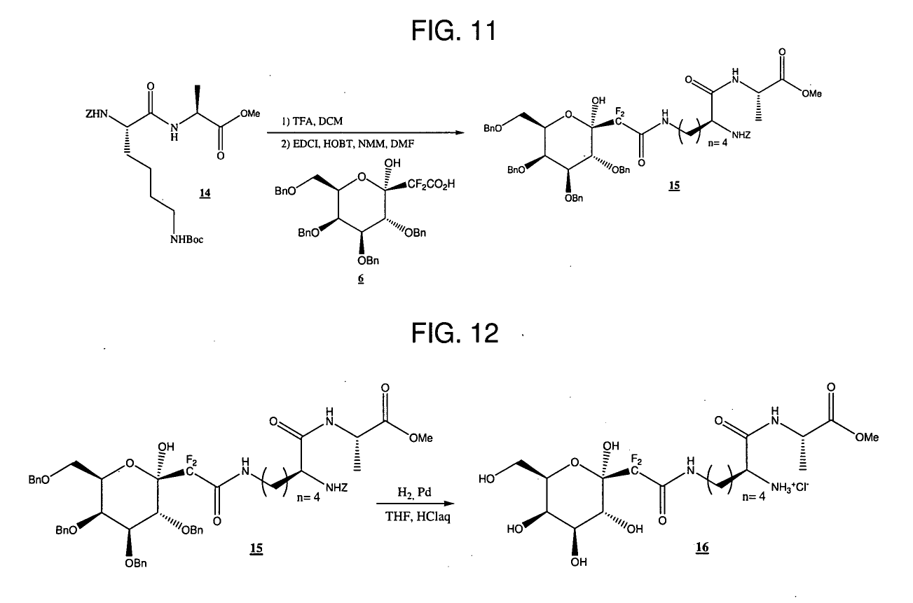

FIG. 11 is a reaction equation to obtain compound 15;

FIG. 12 is a reaction equation to obtain compound 16;

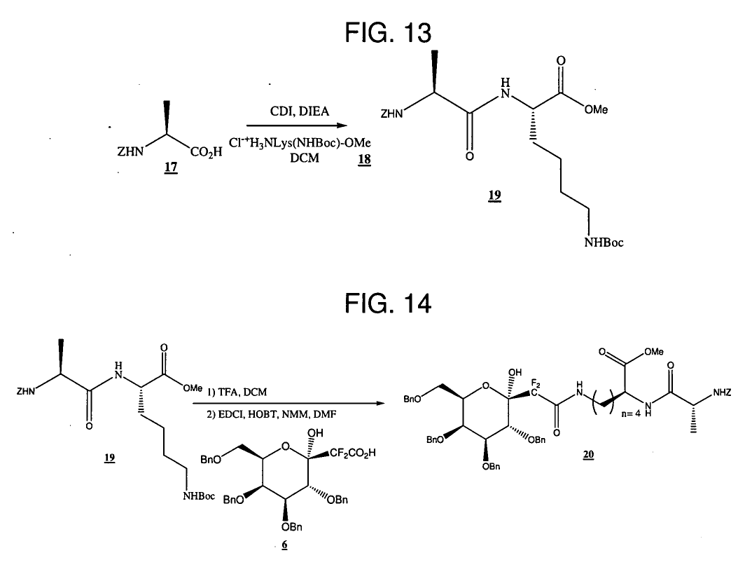

FIG. 13 is a reaction equation to obtain compound 19;

FIG. 14 is a reaction equation to obtain compound 20;

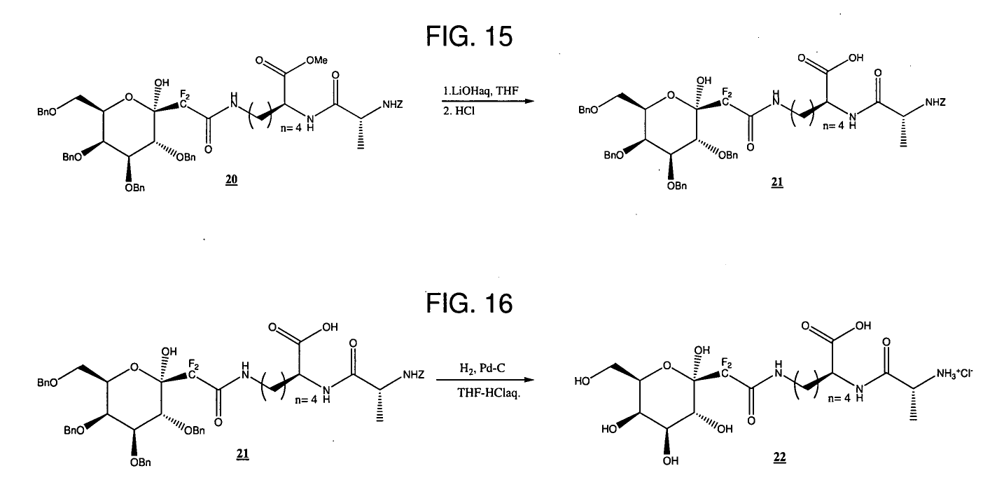

FIG. 15 is a reaction equation to obtain compound 21;

FIG. 16 is a reaction equation to obtain compound 22;

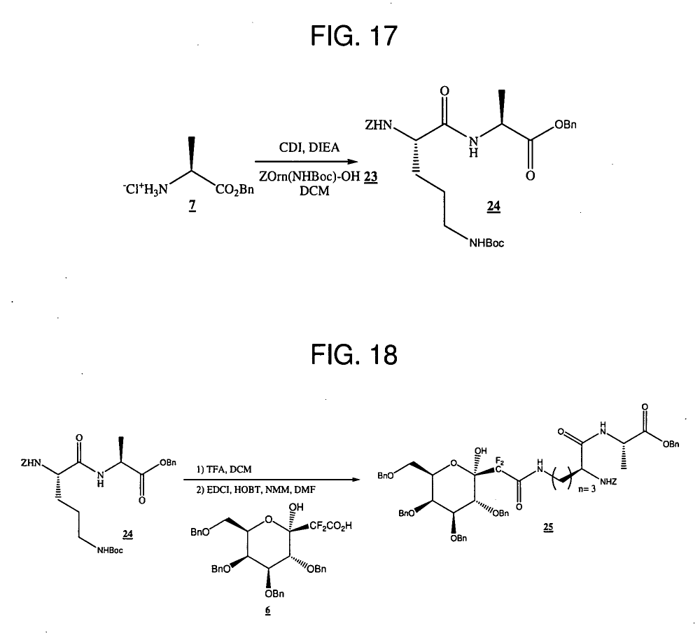

FIG. 17 is a reaction equation to obtain compound 24;

FIG. 18 is a reaction equation to obtain compound 25;

FIG. 19 is a reaction equation to obtain compound 26;

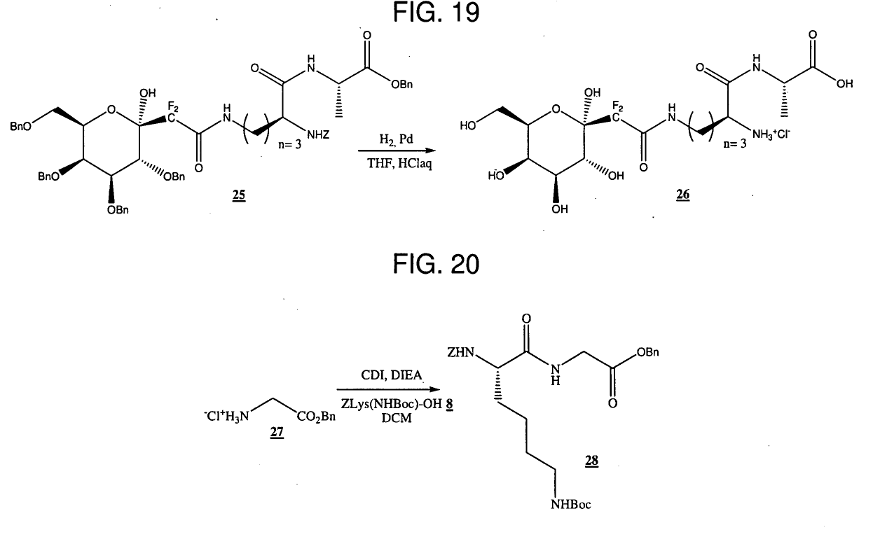

FIG. 20 is a reaction equation to obtain compound 28;

FIG. 21 is a reaction equation to obtain compound 29;

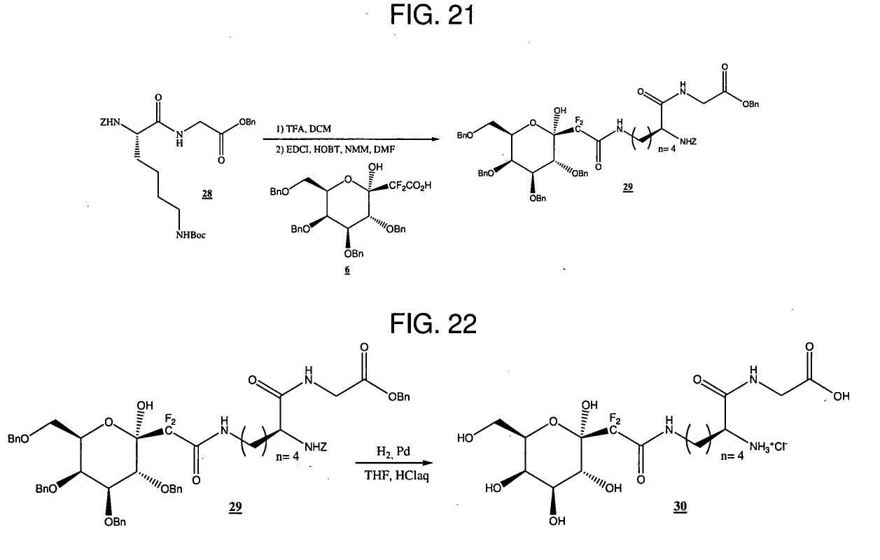

FIG. 22 is a reaction equation to obtain compound 30;

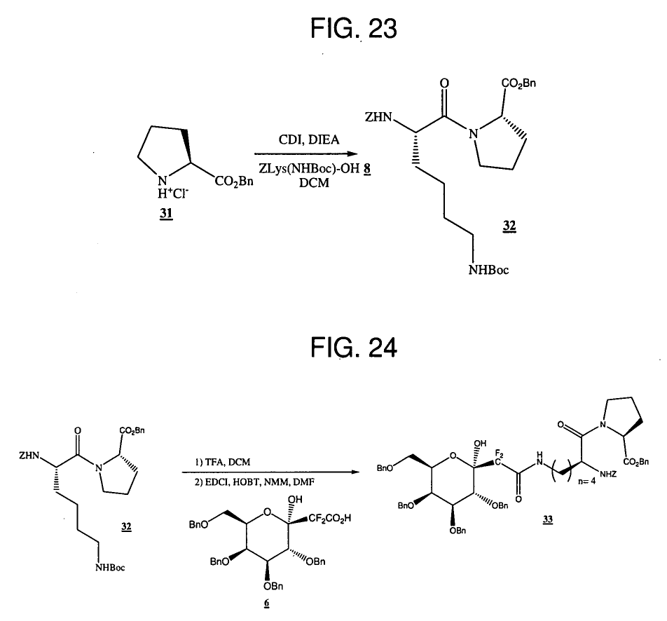

FIG. 23 is a reaction equation to obtain compound 32;

FIG. 24 is a reaction equation to obtain compound 33;

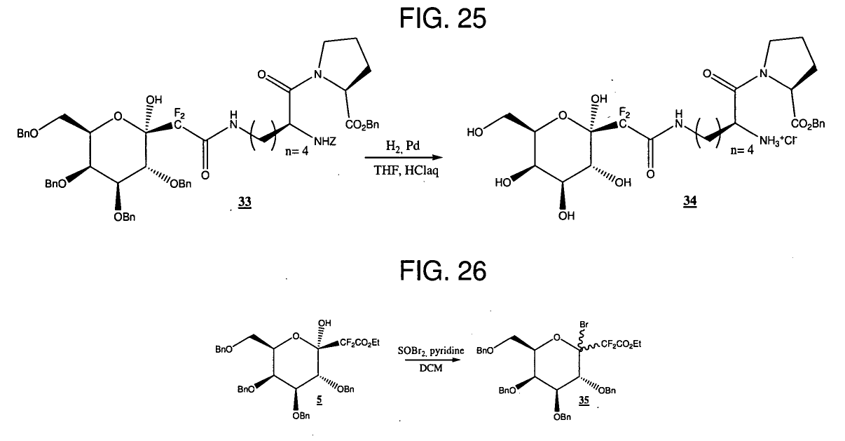

FIG. 25 is a reaction equation to obtain compound 34;

FIG. 26 is a reaction equation to obtain compound 35;

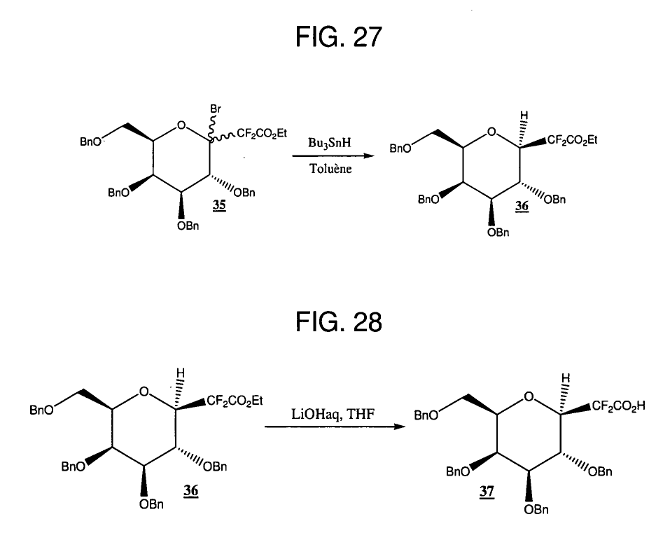

FIG. 27 is a reaction equation to obtain compound 36;

FIG. 28 is a reaction equation to obtain compound 37;

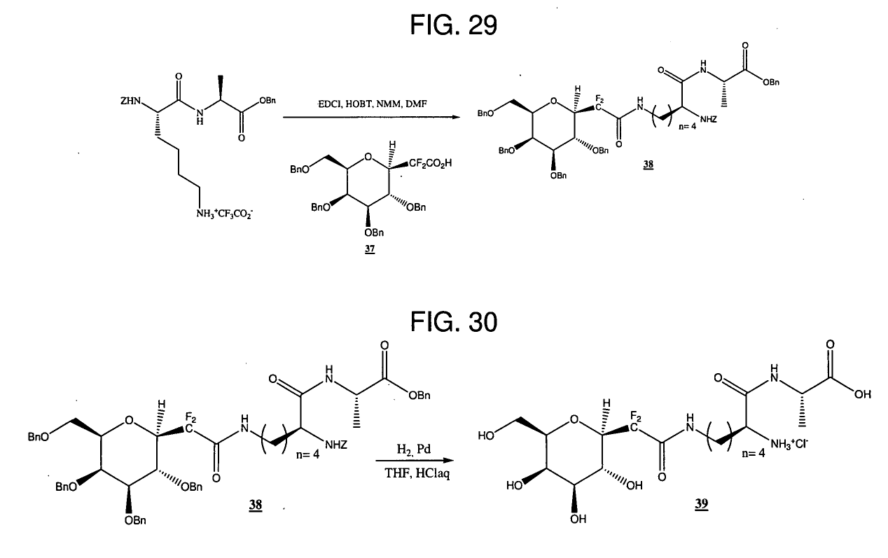

FIG. 29 is a reaction equation to obtain compound 38;

FIG. 30 is a reaction equation to obtain compound 39;

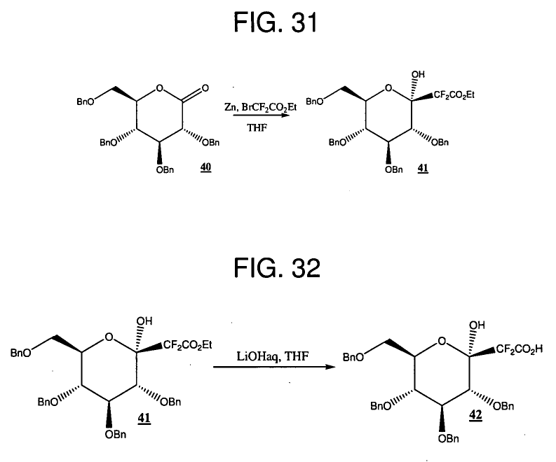

FIG. 31 is a reaction equation to obtain compound 41;

FIG. 32 is a reaction equation to obtain compound 42;

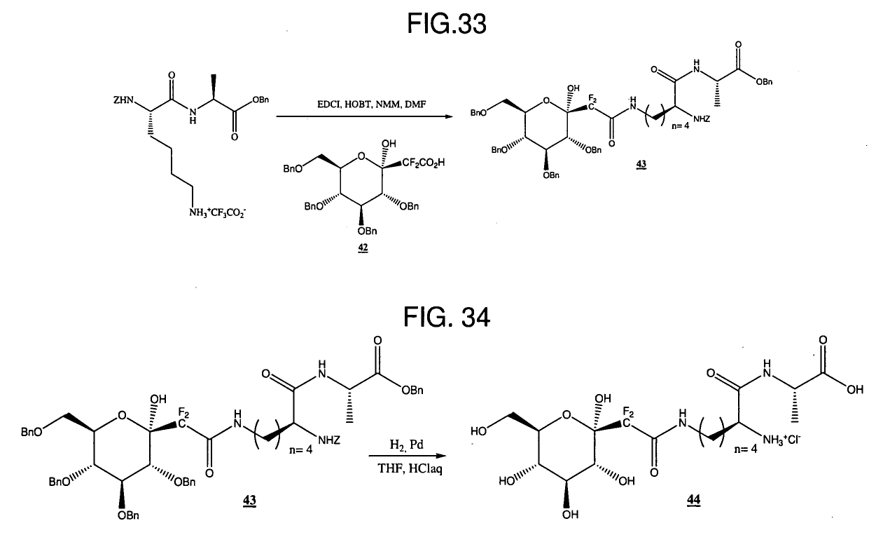

FIG. 33 is a reaction equation to obtain compound 43;

FIG. 34 is a reaction equation to obtain compound 44;

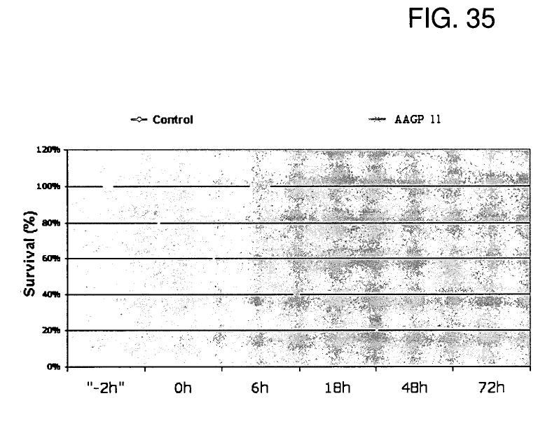

FIG. 35 is a representation of the effects of compound 11 on UV-treated adult skin fibroblasts;

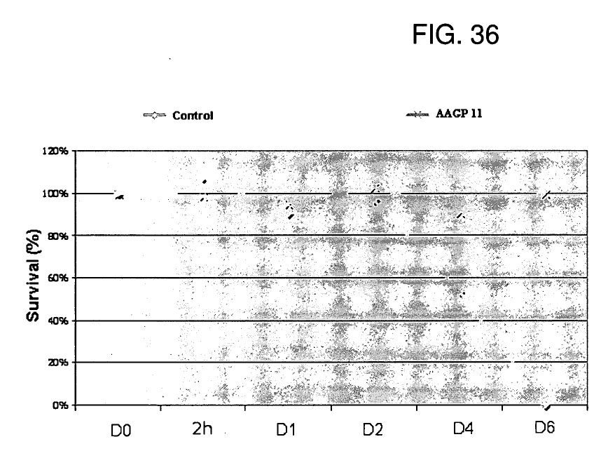

FIG. 36 is a representation of the effects of compound 11 on adult skin fibroblasts at −3° C.;

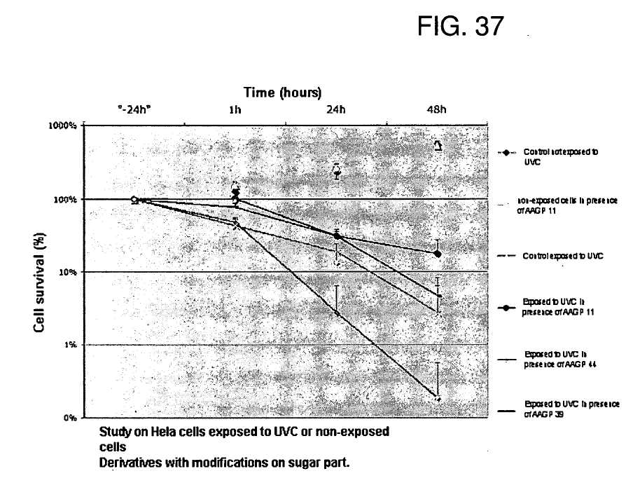

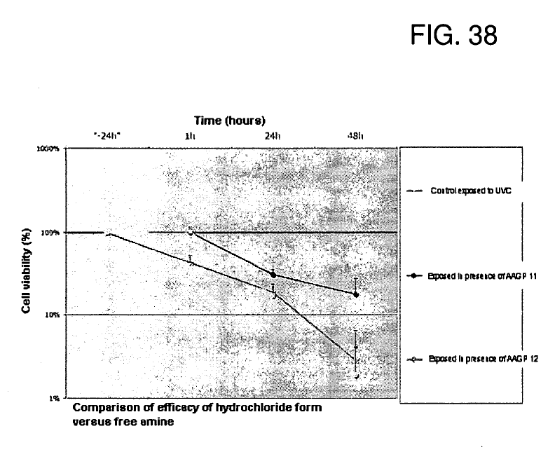

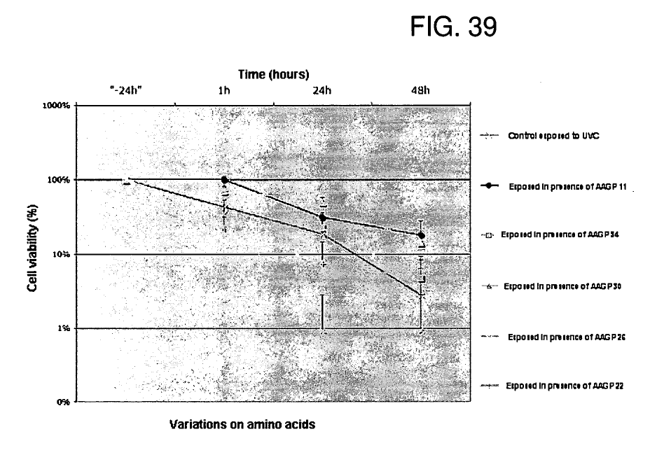

FIGS. 37, 38 and 39 are representations of the effects of the various derivatives on the survival of HELA cells subjected to UVC.

The abbreviations encountered are defined as follows:

eq.: equivalent g: gram Hz: Hertz

mg: milligram MHz: megaHertz min.: minute

mL: millilitre mmol: millimole μmol: micromole

nmol: nanomole app: apparent

The characteristics of the apparatuses used to perform the analyses of all the compounds described in the present application are specified below:

The ¹H, ¹³C, ¹⁹F NMR spectra were recorded on BRUKER DPX 300 and DPX 600 spectrometers. For ¹H and ¹³C NMR, tetramethylsilane is used as the internal reference. For ¹⁹F NMR, the external reference is fluorotrichloromethane CFCl₃. The chemical shifts are expressed in parts per million (ppm), the coupling constants J in Hertz (Hz).

The following abbreviations were used:

s for singlet, bs for a broad singlet, d for doublet, t for triplet, q for quadruplet, m for multiplet or mass, dd for doublet doublet, etc.

The mass spectra were obtained on a Micromass TOF-SPEC, E 20 kV, α-cyano type spectrophotometer for the Maldi ionisation and JEOL AX500, 3 kV, Canon FAB JEOL, Xe, 4 kV, limit current 10 μA, Gly-NBA 50:50 for the FAB ionisation.

The column chromatography separations are performed under slight pressure following the chromatography techniques on Kieselgel 60 silica (230-400 Mesh, Merck).

Monitoring is performed by means of thin layer chromatography (TLC) with Kieselgel 60F-254-0.25 mm plates. The front ratio (Rf) refers to the ratio of the migration distance of a compound on a given substrate on the migration distance of an eluent.

### Synthesis of Compound 2 (FIG. 1)

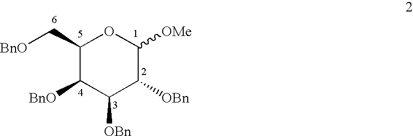

In a flask in an inert atmosphere containing methyl-D-galactopyranoside 1 (5 g; 26 mmol; 1 eq.) and tetrabutylammonium iodide nBu₄NI (500 mg; 1.3 mmol; 0.05 eq.) in dimethylformaldehyde DMF (250 mL), sodium hydride NaH (3.7 g; 0.15 mol; 6 eq.) is introduced in small portions. Benzyl bromide BnBr (18 mL; 0.15 mol; 6 eq.) is then added and the mixture is left under stirring for at least 24 hours.

The medium is hydrolysed with water. The aqueous phase is then extracted three times with ether. The organic phases are then pooled, washed several times with water, dried on magnesium sulphate, filtered and evaporated.

The product obtained in this way is purified by means of chromatography on a silica column with a cyclohexane/ethyl acetate mixture in proportions of nine to one as the eluent. After concentrating the collected fractions, the product 2 is presented in the form of white crystals with a 95% yield by weight.

C₃₅H₃₈O₆ M=554.67 g.mol⁻¹

Rf: 0.38 (cyclohexane/ethyl acetate 8/2).

### Synthesis of Compound 3 (FIG. 2)

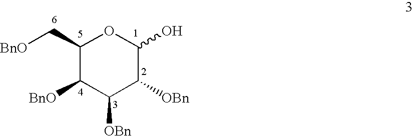

In a flask containing 1-O-Methyl-2,3,4,6-Tetra-O-Benzyl-D-galactopyranose 2 (5.5 g; 9.92 mmol) in 80 mL of acetic acid, 11 mL of sulphuric acid H₂SO₄ is added at a molar concentration of 3M. The reaction medium is heated at 100° C. for one hour. The solution is then diluted in 100 mL of cold water.

The mixture is extracted four times with 100 mL of toluene. The organic phases are pooled and washed with 100 mL of water, 100 mL of a saturated sodium hydrogen carbonate solution NaHCO₃ and finally with 100 mL of water. The organic phase is then dried on magnesium sulphate, filtered and concentrated.

The product obtained in this way is purified by means of chromatography on a silica column using a cyclohexane/ethyl acetate mixture in proportions of 8.5 to 1.5 as the eluent. After concentrating the fractions collected, the product 3 is in the form of white crystals with a yield by weight of 75%.

C₃₄H₃₆O₆ M=540.65 g.mol⁻¹

Rf: 0.65 (cyclohexane/ethyl acetate 6/4).

### Synthesis of Compound 4 (FIG. 3)

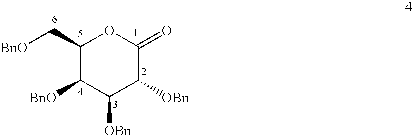

In a flask in an inert atmosphere, containing 2,3,4,6-Tetra-O-Benzyl-D-Galactopyranose 3 (4 g; 7.4 mmol), dimethylsulphoxide DMSO (25.6 mL) and acetic anhydride Ac₂O (16.8 mL) are introduced. The mixture is left under stirring for 12 hours.

Water is then added and the aqueous phase is extracted three times with ether. The organic phases are pooled and washed with a saturated sodium hydrogen carbonate solution NaHCO₃ and water. The organic phase is then dried on magnesium sulphate, filtered and concentrated.

The product is then purified by means of chromatography on a silica column using a cyclohexane/ethyl acetate mixture in proportions of eight to two as the eluent. After concentrating the fractions collected, lactone 4 is presented in the form of a colourless oil with an 82% yield by weight.

C₃₄H₃₄O₆ M=538.63 g.mol⁻¹

Rf: 0.61 (cyclohexane/ethyl acetate 8/2).

¹H NMR (CDCl₃, 300 MHz)

3.6 (m, 2H, H6); 3.8 (dd, 2.1-9.6, 1H, H3); 4.1 (s, 1H, H4); 4,2 (m, 1H, H5); 4.4-5.1 (m, 9H, H2; 4OCH₂Ph); 7.2 (m, 20H, H ar.)

¹³C NMR (CDCl₃, 75.5 MHz)

67.4 (C6); 72.4 (C5); 72.6 (OCH₂Ph); 73.5 (OCH₂Ph); 74.5 (C4); 75.1 (OCH₂Ph); 77.1 (C2); 79.9 (C3); 127.4-128.3 (Car.); 137.2; 137.3; 137.6 (quat. Car.); 169.8 (CO).

α_{D}=+79.0° (c=1; CHCl₃)

### Synthesis of Compound 5 (FIG. 4)

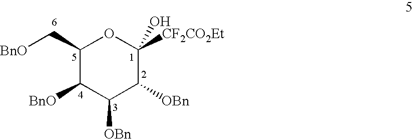

In a flask in an inert atmosphere containing Zn (1.7 g; 26 mmol; 7 eq.) previously activated and pickled in tetrahydrofuran THF (30 mL) in a reflux, a mixture consisting of lactone 4 (2 g; 3.7 mmol; 1 eq.) and ethyl bromodifluoroacetate (1.42 mL; 11 mmol; 3 eq.) in THF (30 mL) is added drop by drop. The reaction is left to reflux for 3 hours. After returning to ambient temperature, the zinc is filtered, and a 1N hydrochloric acid solution HCl (60 mL) followed by dichloromethane (6 mL) are added to the reaction medium.

The aqueous and organic phases are separated and the aqueous phase is extracted a further two times with dichloromethane. The organic phases are pooled, dried on magnesium sulphate, filtered and concentrated.

The product is then purified by means of chromatography on a silica column using a cyclohexane/ethyl acetate in proportions of eight to two as the eluent. After concentrating the collected fractions, the product 5 is presented in the form of white crystals with an 82% yield by weight.

C₃₈H₄₀F₂O₈ M=662.72 g.mol⁻¹

Rf: 0.35 (cyclohexane/ethyl acetate 8/2).

¹⁹F NMR (CDCl₃; 282.5 MHz)

−118.4 (d, J_{F—F}=256 Hz); −120.2 (d, J_{F—F}=256 Hz).

¹H NMR (CDCl₃, 300 MHz)

1.1 (t; 7.2; 3H; CH₃); 3.4-3.5 (m; 2H; H6); 3.7-3.8 (dd; 2.5-9.5; 1H; H3); 3.8 (d; 2; 1H; H4); 4-4.1 (m; 3H; H5; CH₂); 4.25-4.85 (m; 9H; H2; 4OCH₂Ph); 7.2 (m; 20H; Har).

¹³C NMR (CDCl₃, 75.5 MHz)

14.2 (CH₃); 63.6 (CH₂); 68.6 (C6); 71.7 (C5); 73.2 (OCH₂Ph); 73.9 (OCH₂Ph); 74.1 (C4); 74.9 (OCH₂Ph); 75.1 (C2); 75.8 (OCH₂Ph); 81.2 (C3); 96.9 (t, 27 Hz, C1); 113 (t, 264 Hz, CF₂); 128.0-128.9 (Car.); 138.2; 138.3; 138.6; 139.1 (quat. Car.); 163.3 (t, 31 Hz, CO₂Et).

α_{D}=+52.6° (c=1; CHCl₃)

### Synthesis of Compound 6 (FIG. 5)

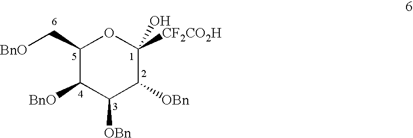

In a flask containing ester 5 (0.5 g; 1.75 mmol, 1 eq.) in THF (5 mL) an aqueous lithine solution LiOH (84 mg; 3.5 mmol, 2 eq.) solubilised in a minimum quantity of water is added. The mixture is left for 12 hours under stirring and taken up in ethyl acetate. The mixture is acidified with an aqueous 1N hydrochloric acid solution and extracted several times with ethyl acetate. The organic phases are pooled, dried on magnesium sulphate, filtered and concentrated.

Compound 6 is obtained in the form of a white oil with a quantitative yield.

C₃₆H₃₆F₂O₈ M=634.66 g.mol⁻¹

¹⁹F NMR (CDCl₃, 282.5 MHz)

−117.3 (d, J_{F—F}=259 Hz); −119.0 (d, J_{F—F}=259 Hz).

¹H NMR (CDCl₃, 300 MHz)

3.2 (dd; 4.5 Hz and 9.8 Hz; 1H; H6); 3.5 (dd; 7.7 Hz and 9.8 Hz; 1H; H6); 3.7 (d; 2 Hz; 1H; H4); 3.8 (dd; 2.6 Hz and 9.5 Hz; 1H; H3); 4 (dd; 4.5 Hz and 7.7 Hz; 1H, H5); 4.3-4.9 (m; 9H; H2; 4OCH₂Ph); 7.2 (m; 20H; Har).

¹³C NMR (CDCl₃, 75.5 MHz)

69.4 (C6); 71.7 (C5); 73.5 (OCH₂Ph); 74.0 (OCH₂Ph); 74.1 (C4); 75.0 (OCH₂Ph); 75.1 (C2); 75.9 (OCH₂Ph); 80.8 (C3); 95.4 (t, 27 Hz, C1); 112.5 (t, 260 Hz, CF₂); 127.8-129.0 (Car.); 137.6; 138.0; 138.1 (quat. Car.); 163.1 (t; 30 Hz; CO₂H).

### Synthesis of Compound 9 (FIG. 6)

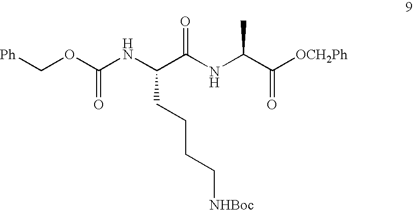

In a flask in an inert atmosphere containing 7-Lysine(Boc)-OH 8 (2 g; 5.26 mmol; 1 eq.) in dichloromethane (40 mL), carbonyldiimadozole CDI (878 mg; 5.42 mmol; 1.03 eq.) is added. The reaction is left under stirring for one hour. To this mixture, a solution prepared in an inert atmosphere, consisting of Cl⁻⁺H₃N-AlanineOBn 7 (1.13 g; 5.26 mmol; 1 eq.), and diisopropylethylamine DIEA (1.92 mL; 11.04 mmol; 2 eq.) in dichloromethane (40 mL) is added. The reaction is continued for 24 hours and hydrolysed with water and extracted three times with dichloromethane. The organic phases are pooled, dried on magnesium sulphate, filtered and concentrated.

The mixture is then purified by means of chromatography on a silica column using a cyclohexane/ethyl acetate mixture in proportions of seven to three as the eluent. After concentrating the collected fractions, the product 9 is presented in the form of a pale yellow solid with a yield by weight of 66%.

C₃₂H₃₇N₃O₇ M=544.5 g.mol⁻¹

¹H NMR (CDCl₃, 300 MHz)

1.3 (m; 16H; CH₃; (CH₃)₃C; 2CH₂); 1.6-1.7 (m; 2H; CH₂); 3.0 (m; 2H; NHCH₂); 4.1 (m; 1H; CHLys); 4.5 (m; 1H; CHAla); 4.6 (m; 1H; NH); 5.0 (s; 2H; OCH₂Ph); 5.1 (m; 1H; ZNH); 5.5 (d; 7 Hz; 1H; NH); 6.8 (d; 6.5 Hz; 1H; NH); 7.3 (m; 5H; Har.).

¹³C NMR (CDCl₃, 75.5 MHz)

18.4 (CH₃); 22.6 (CH₂); 28.8 ((CH₃)₃C); 29.8 (CH₂); 32.6 (CH₂); 40.1 (NCH₂); 48.6 (CHAla); 54.9 (CHLys); 67.4 and 67.5 (2OCH₂Ph); 80.2 ((CH₃)₃C); 128.5-129 (Car.); 135.6 and 136.6 (quat. Car.); 156.6 (CO(Boc) and CO(Z)); 171.8 and 172.9 (CONH and CO₂Et).

### Synthesis of Compound 10 (FIG. 7)

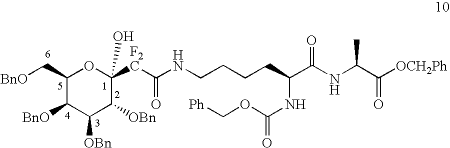

### Peptide Deprotection

In a flask in an inert atmosphere containing the peptide 9 (1.8 g; 3.36 mmol; 1 eq.) in dichloromethane (40 mL), trifluoroacetic acid TFA (5 mL; 67.2 mmol; 20 eq.) is introduced. The mixture is left to react for 12 hours and the reaction medium is then concentrated. Four to five co-evaporations with toluene are performed to obtain the deprotected product with a quantitative yield.

### Coupling

In a flask in an inert atmosphere containing the acid 6 (1.38 g; 2.18 mmol; 1.05 eq.), the peptide deprotected above (1.15 g; 2.08 mmol; 1 eq.), 1-hydroxybenzotriazole HOBT (0.336 g; 2.5 mmol; 1.2 eq.) and N-methylmorpholine NMM (0.57 mL; 5.2 mmol; 2.5 eq.) in DMF (35 mL), EDCI (0.478 g; 2.49 mmol; 1.2 eq.) is added. The reaction is left under stirring for 24 hours, and the solvent is evaporated and the medium is taken up with dichloromethane. The medium is then extracted twice with 1M HCl (2*100 mL). The organic phase is collected, dried on magnesium sulphate, filtered and concentrated.

The mixture is then purified by means of chromatography on a silica column with a cyclohexane/ethyl acetate mixture as the eluent. After concentrating the collected fractions, the product 10 is presented in the form of a white solid with an 83% yield by weight.

C₆₀H₆₅F₂N₃O₁₂ M=1058.17 g.mol⁻¹

Rf: 0.48 (Cyclohexane/ethyl acetate 5/5).

¹⁹F NMR (CDCl₃, 282.5 MHz)

−116.9 (d, J_{F—F}=259 Hz); −121.7 (d, J_{F—F}=259 Hz).

¹H NMR (CDCl₃, 300 MHz)

1.2-1.4 (m; 7H; 1CH₃; 2CH₂); 1.5 (m; 1H; CH₂); 1.6 (m; 1H; CH₂); 3.0 and 3.2 (2m; 2H; NHCH₂); 3.4 (m; 2H; H6); 3.8-3.9 (m; 2H; H3; H4); 4.0 (m; 2H; CHNH (Lys); H5); 4.2-4.9 (m; 9H; 4OCH₂Ph; CH (Ala)); 4.2 (d; 9 Hz; 1H; H2); 5.0 (s; 2H; OCH₂Ph); 5.1 (m; 1H; ZNH); 5.4 (d; 7.8; 1H; NH); 6.5 (d; 7.2; 1H; NH); 6.7 (s; 1H; NH); 7.2 (m; 30H; Har.).

¹³C NMR (CDCl₃, 75.5 MHz)

18.3 (CH₃); 22.4 (CH₂); 28.7 (CH₂); 32.5 (CH₂); 39.3 (CH₂N); 48.6 (CH Ala); 54.9 (NCH Lys); 67.4 and 67.6 (2OCH₂Ph); 68.7 (C6); 71.2 (C5); 73.5 and 73.8 (2OCH₂Ph); 74.4 (C4); 75.0 (OCH₂Ph; C2); 75.8 (OCH₂Ph); 80.9 (C3); 97.2 (t, 27 Hz, C1); 113 (t, CF₂); 127.9-129 (Car.); 135.7; 136.7; 138.2; 138.3; 138.7; 139.0 (quat. Car.); 156.0 (CO(Z)); 163.7 (t, 28 Hz, CF₂CONH); 172.0 (CONH); 173.0 (CO₂Bn).

### Synthesis of Compound 11 (FIG. 8)

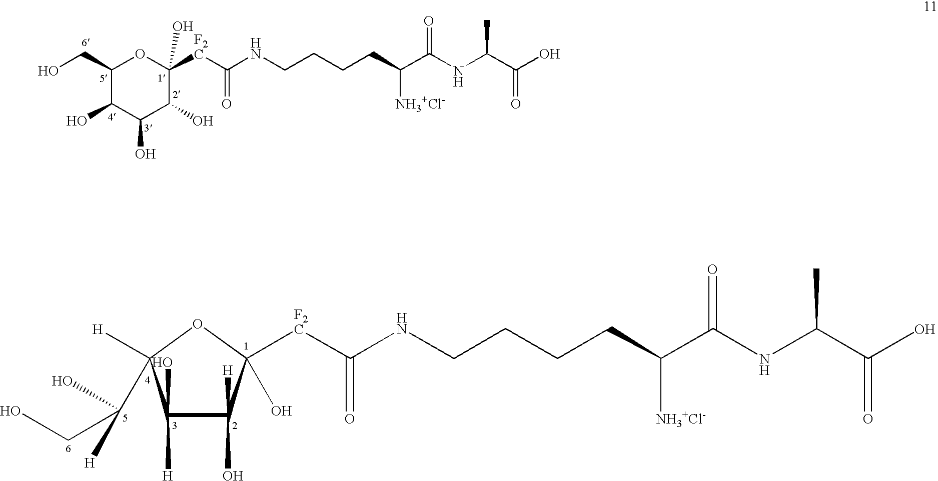

A flask containing the initial product 10 (150 mg; 0.133 mmol) in a mixture of tetrahydrofuran THF (3 mL) and 1N HCl (2 mL) in the presence of a spatula tip of palladium on carbon Pd/C is placed in a hydrogen atmosphere. The mixture is left under stirring overnight and filtered on a Millipore® filter. The mixture is then concentrated to obtain the product 11 in the form of an orangey yellow solid with a 92% yield.

C₁₇H₃₀ClF₂N₃O₁₀ M=509.88 g.mol⁻¹

¹⁹F NMR (D₂O, 282.5 MHz)

−119.4 (d, J_{F—F}=256 Hz); −120.6 (d, J_{F—F}=256 Hz); −121.0 (d, J_{F—F}=257 Hz); −122.0 (d, J_{F—F}=257 Hz).

¹H NMR (D₂O, 300 MHz)

1.4 (d; 7.3 Hz; CH₃); 1.4 (m; 2H; CH₂); 1.5 (m; 2H; CH₂); 1.8 (m; 2H; CH₂); 3.2 (m; 2H; NHCH₂); 3.5-3.6 (m; 2H6′,2H6); 3.7 (m; H4 H5); 3.8 (dd, 3.4 Hz and 9.9 Hz, H3′) 3.9 (m; CH,H4′,H5′,H2′); 4.0 (t, 8.2 Hz, H3); 4.3 (m; CH); 4.36 (d, 8.2 Hz, H2).

¹³C NMR (D₂O, 75.5 MHz)

16.2 (CH₃); 21.5 (CH₂); 28.0 (CH₂); 30.7 (CH₂); 39.3 (CH₂N); 49.1 (CH); 53.2 (NCH); 61.1 (C6′); 62.6 (C6); 67.0 (C5′); 68.9 (C4′); 70.6 (C3′); 70.9 (C5); 72.5 (C2′); 73.9 (C3); 75.5 (C2); 80.2 (C4); 169.9 and 176.3 (CO).

### Synthesis of Compound 12 (FIG. 9)

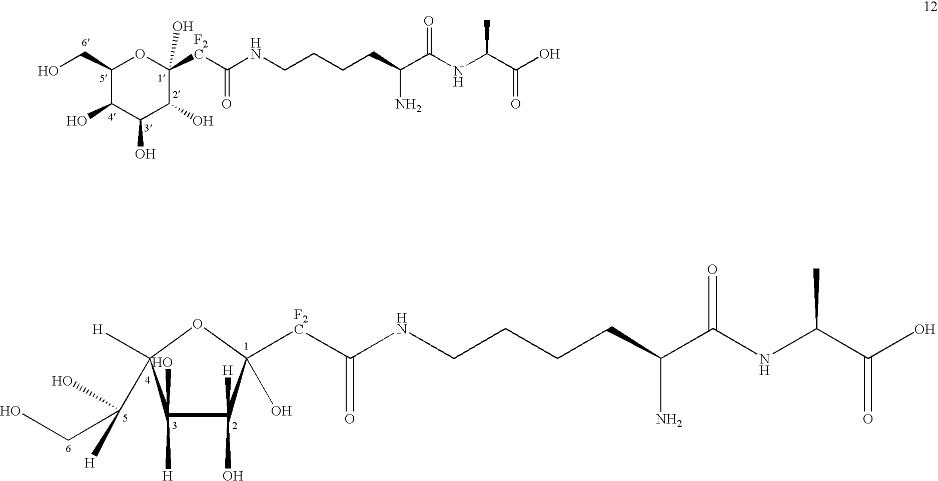

In a flask, the compound 10 (0.81 mmol) is dissolved in a water/tetrahydrofuran mixture (1:1; 20 mL) and palladium on carbon and placed in a hydrogen atmosphere. The mixture is stirred for 2 days at ambient temperature. The reaction mixture is filtered and concentrated. The unprocessed product is taken up with dichloromethane (20 mL) which is removed, and then with water (10 mL) which is filtered. The aqueous phase is then concentrated to leave the desired product as a white solid with a 54% yield.

C₁₈H₃₁F₂N₃O₁₀ M=473.42 g.mol⁻¹

¹⁹F NMR (D₂O, 282 MHz)

−119.3 (1F, d, ²J_{F—F}255.5), 120.4 (1F, d, ²J_{F—F}255.5)

−120.7 (1F, d, ²J_{F—F}256.6), 121.8 (1F, d, ²J_{F—F}255.5)

¹H NMR (D₂O, 300 MHz)

1.31 (3H, d, ³J_{H11-H9} 7.3, CH₃), 1.37-1.45 (2H, m, H CH₂), 1.52-1.61 (2H, m, CH₂), 1.74-1.87 (2H, m, CH₂), 3.25-3.31 (2H, m, NCH₂), 3.57-3.75 (4H, m, 2H6′,2H6,H4,H5), 3.82 (dd, ³J_{H—H} 3.2, ³J_{H—H} 9.7, H3′), 3.95-4.02 (CH,H4′,H5′,H2′), 4.08-4.17 (m, CH,H3), 4.39 (d, ³J_{H—H} 8.3,H2),

¹³C NMR (D₂O, 75 MHz)

17.5 (CH₃), 21.9 (CH₂), 28.0 (CH₂) 31.8 (CH₂), 39.5 (NCH₂), 51.5 (CH), 53.8 (CH), 61.1 (C6′); 62.6 (C6); 67.1 (C5′); 68.9 (C4′); 70.6 (C3′); 70.9 (C5); 72.4 (C2′); 74.0 (C3); 75.6 (C2); 80.2 (C4); 96.1 (d, ²J_{C1′-F}25.7), 98.9 (t, ²J_{C1′-F}28.6) (C1 and C1′), 114.0 (t, ²J_{C7′-F}256.5, CF₂), 163.9 (t, ²J_{C8′-F} 28.0, CF₂CO), 169.9 and 176.3 (CO).

Mass spectrometry: ESI+: 496 (MH+Na)+, 474 (MH)+.

### Synthesis of Compound 14 (FIG. 10)

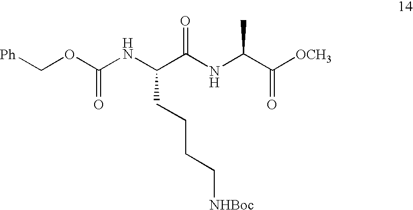

In a flask in an inert atmosphere containing Z-Lysine(NHBoc)-OH 8 (5 g; 13.14 mmol; 1 eq.) in dichloromethane (50 mL), carbonyldiimadozole CDI (2.56 g; 15.8 mmol; 1.2 eq.) is added. The reaction is left under stirring for one hour. To this mixture, a solution prepared in an inert atmosphere, consisting of Cl⁻⁺H₃N-AlanineOMe 13 (1.84 g; 13.14 mmol; 1 eq.), and diisopropylethylamine DIEA (4.8 mL; 27.6 mmol; 2.1 eq.) in dichloromethane (50 mL) is added. The reaction is continued for 24 hours and a 1N hydrochloric acid solution (50 mL) is added, and the mixture is extracted with dichloromethane (3×50 mL). The organic phases are washed with a saturated NaCl solution (100 mL), dried on MgSO₄ and concentrated in a vacuum to leave the compound 14 in the form of a white solid which is used directly following the synthesis with a 99% yield.

C₂₃H₃₅N₃O₇ M=465.5 g.mol⁻¹

Rf=0.43, eluent: cyclohexane/ethyl acetate (1:1).

¹H NMR (CDCl₃, 300 MHz)

1.30 (d, 3H, ³J_{H12-H9} 7.4, CH₃;), 1.33-1.45 (m, 12H, (CH₃)₃C; CH₂), 1.52-1.69 (m, 2H, CH₂), 1.76-1.82 (m, 1H, CH₂), 3.00-3.08 (m, 2H, NCH₂), 3.66 (s, 3H, CH₃), 4.25 (m, 1H, (CH), 4.48 (qt, 1H, ³J_{H9-H12} 7.4, (CH), 5.03 (s, 3H, OCH₂Ph, NH), 6.03 (d, 1H, ³J_{NH—H6} 7.8, NH), 7.22-7.31 (m, 6H, H_{Ar}, NH).

¹³C NMR (CDCl₃, 75.5 MHz)

18.0 (CH₃); 22.6 (CH₂); 28.8 ((CH₃)₃C); 29.7 (CH₂); 32.7 (CH₂); 40.2 (NCH₂); 48.4 (CH(Ala)); 52.7 (CH(Lys)); 67.2 (OCH₂Ph); 79.3 ((CH₃)₃C); 128.3; 128.4 (2C); 128.8 (2C) (Car.); 136.6 (quat. Car.); 156.6 and 156.7 (CO(Boc) and CO(Z)); 172.4 and 173.6 (CONH and CO₂Et).

### Synthesis of Compound 15 (FIG. 11)

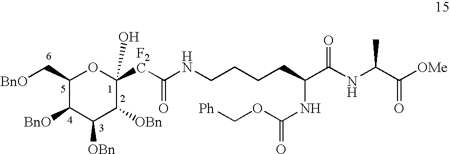

### Peptide Deprotection

In a flask in an inert atmosphere containing peptide 14 (6 g; 13 mmol; 1 eq.) in dichloromethane (100 mL), trifluoroacetic acid TFA (20 mL; 263 mmol; 20 eq.) is introduced. The mixture is left to react for 12 hours and reaction mixture is then concentrated. Four to five co-evaporations with toluene are performed to obtain the deprotected product with a quantitative yield.

### Coupling

In a flask in an inert atmosphere containing acid 6 (2.15 g; 2.49 mmol; 1.0 eq.), the peptide deprotected above (1.20 g; 2.49 mmol; 1 eq.), 1-hydroxybenzotriazole HOBT (0.37 g; 2.75 mmol; 1.1 eq.) and N-methylmorpholine NMM (0.83 g; 8.22 mmol; 3.3 eq.) in DCM (100 mL), EDCI (0.53 g; 2.75 mmol; 1.1 eq.) is added after 15 minutes. The reaction is left under stirring for 24 hours and water (100 mL) is then added along with dichloromethane, and the aqueous phase is extracted with DCM (3×100 mL). The organic phases are washed with a saturated NaCl solution (200 mL), dried on MgSO₄ and concentrated in a vacuum to leave a beige solid.

The residue is purified by means of chromatography on silica gel with a cyclohexane/AcOEt (1:1) as the eluent to produce the pure product 15 in the form of a white solid with a 38% yield.

C₅₄H₆₁F₂N₃O₁₂ M=982.07 g.mol⁻¹

Rf=0.27, eluent: cyclohexane/ethyl acetate (1:1).

¹⁹F NMR (CDCl₃, 282 MHz)

−117.0 (1F, d, ²J_{F—F} 258.0), −121.9 (1F, d, ²J_{F—F} 258.0).

¹H NMR (CDCl₃, 300 MHz)

1.31 (d, 3H, ³J_{H12-H9} 7.1, CH₃), 1.30-1.78 (m, 6H, 3CH₂), 3.05-3.38 (m, 2H, NCH₂), 3.42-3.51 (m, 2H, H6), 3.66 (s, 3H, OCH₃), 3.88 (m, 2H), 4.10 (m, 1H), 4.28 (d, 1H, J 9.0), 4.39-4.55 (m, 4H), 4.68-4.92 (m, 5H), 5.02-5.06 (m, 2H), 5.40 (d, 1H, ³J7.9, NH), 6.35 (d, 1H, ³J7.2, NH), 6.78 (1s, 1H, NH), 7.22-7.29 (m, 25H, H_{Ar}).

### Synthesis of Compound 16 (FIG. 12)

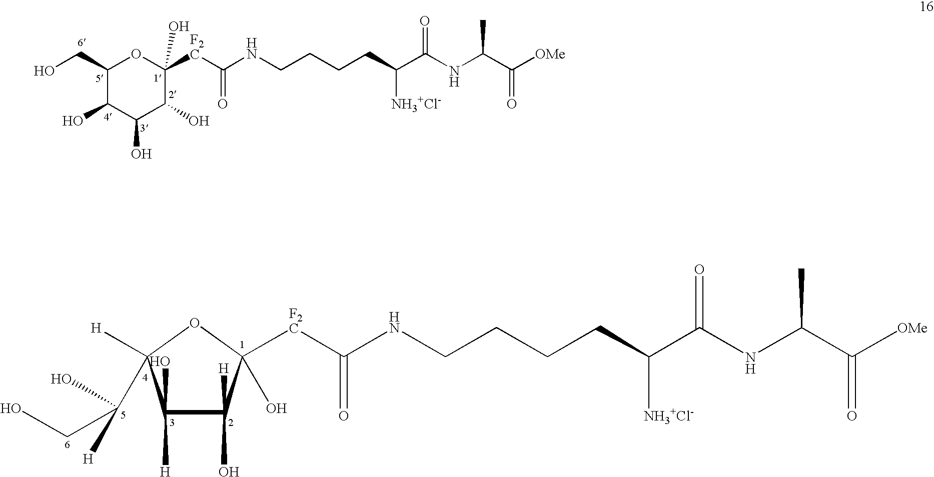

A flask containing the initial product 15 (497 mg; 0.488 mmol ) in a mixture of tetrahydrofuran THF (10 mL) and 1N HCl (1.2 eq.) in the presence of a spatula tip of palladium on carbon Pd/C is placed in a hydrogen atmosphere. The mixture is left under stirring overnight and then filtered on a Millipore® filter. The mixture is then concentrated to obtain the product 16 in the form of a white solid with an 87% yield.

C₁₈H₃₂ClF₂N₃O₁₀ M=523.5 g.mol⁻¹

¹⁹F NMR (D₂O, 282 MHz)

−119.1 (1F, d, ²J_{F—F} 256.4), −120.5 (1F, d, ²J_{F—F} 256.4),

−120.3 (1F, d, ²J_{F—F} 256.4), −121.7 (1F, d, ²J_{F—F} 256.4).

¹H NMR (D₂O, 300 MHz)

1.37-1.48 (m, 5H, CH₃,CH₂), 1.54-1.65 (m, 2H, CH₂), 1.72-1.91 (m, 2H, CH₂), 3.24-3.32 (m, 2H, NCH₂), 3.59-3.85 (m), 3.96-4.02 (m), 4.13 (, 1H, ³J 7.9, H2).

### Synthesis of Compound 19 (FIG. 13)

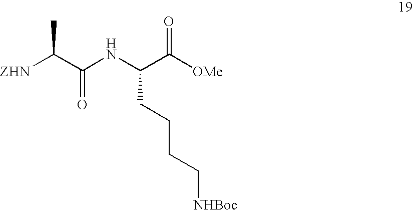

In a flask in an inert atmosphere containing Z-Ala-OH 17 (752 mg; 3.37 mmol; 1.0 eq.) in dichloromethane (15 mL), carbonyldiimadozole CDI (656 g; 4.05 mmol; 1.2 eq.) is added. The reaction is left under stirring for one hour. To this mixture, a solution prepared in an inert atmosphere consisting of Cl⁻⁺H₃N-Lys(NHBoc)OMe 18 (1 g; 3.37 mmol; 1 eq.), and diisopropylethylamine DIEA (1.25 mL; 7.1 mmol; 2.1 eq.) in dichloromethane (15 mL) is added. The reaction is continued for 24 hours. A 1N hydrochloric acid solution (20 mL) is added, and the mixture is extracted with dichloromethane (3×20 mL). The organic phases are washed with a saturated NaCl solution (50 mL), dried on MgSO₄ and concentrated in a vacuum to leave a white solid. The residue is purified on a chromatographic column on silica gel with a cyclohexane/ethyl acetate (1:1) mixture as the eluent in order to isolate the pure product 19 in the form of a white solid with a 72% yield.

C₂₃H₃₅N₃O₇ M=465.5 g.mol⁻¹

Rf=0.25, eluent: cyclohexane/ethyl acetate (1:1).

¹H NMR (CDCl₃, 300 MHz)

1.38 (d, 3H, ³J 6.8, CH_{3;}), 1.25-1.84 (m, 6H, 3CH₂), 1.42 (s, 9H, C(CH₃)₃), 3.02-3.09 (m, 2H, NCH₂), 3.72 (s, 3H, OCH₃), 4.29 (tapp., 6.8 Hz, 1H, CH), 4.54-4.58 (m, 1H, CH), 4.77 (1s, 1H); 5.1 (s, 2H, OCH₂Ph), 5.56 (s, 1H, NH), 6.71 (d, 1H, ³J 7.5, NH), 7.33 (m, 5H, H_{Ar}).

### Synthesis of Compound 20 (FIG. 14)

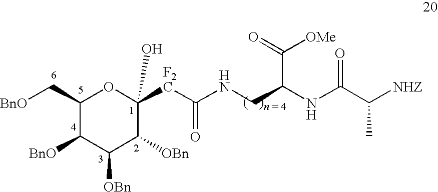

### Peptide Deprotection

In a flask in an inert atmosphere containing peptide 19 (1 eq.) in dichloromethane, trifluoroacetic acid TFA (20 eq.) is introduced. The mixture is left to react for 12 hours and the reaction medium is concentrated. Four to five co-evaporations with toluene are performed to obtain the deprotected product with a quantitative yield.

### Coupling

In a flask in an inert atmosphere containing acid 6 (1.26 g; 2.0 mmol; 1.0 eq.), the peptide deprotected above (0.95 g; 2.0 mmol; 1 eq.), 1-hydroxybenzotriazole HOBT (0.29 g; 2.25 mmol; 1.1 eq.) and N-methylmorpholine NMM (0.28 g; 3 mmol; 1.5 eq.) in DMF (35 mL), EDCI (0.44 g; 2.0 mmol; 1.1 eq.) is added after 15 minutes. The reaction is left under stirring for 24 hours and concentrated. A 1N HCl solution (20 mL) is added along with dichloromethane, and the aqueous phase is extracted with DCM (3×30 mL). The organic phases are washed with a saturated NaCl solution (50 mL), dried on MgSO₄ and concentrated in a vacuum.

The residue is purified by means of chromatography on silica gel with a cyclohexane/AcOEt mixture (1:1) as the eluent to produce the pure product 20 in the form of a white solid with a 33% yield.

C₅₄H₆₁F₂N₃O₁₂ M=982.07 g.mol⁻¹

Rf=0.30, eluent: cyclohexane/ethyl acetate (1:1).

¹⁹F NMR (CDCl₃, 282 MHz)

−117.8 (1F, d, ²J_{F—F} 259.2), −120.3 (1F, d, ²J_{F—F} 259.2).

¹H NMR (CDCl₃, 300 MHz)

1.32 (d, 3H, ³J 7.0, CH₃), 1.35-1.80 (m, 6H, 3CH₂), 2.92-3.18 (m, 1H, NCH₂), 3.25-3.36 (m, 1H, NCH₂), 3.51 (d, 6.5 Hz, 2H, H6), 3.71 (s, 3H, OCH₃), 3.88-3.96 (m, 2H, H3, H4), 4.13 (t, 2.3 Hz, 1H, H5), 4.15-4.26 (m, 1H, CH), 4.33 (d, 10 Hz, 1H, H2); 4.42 (d, 2H, OCH₂Ph), 4.45-4.52 (m, 1H, CH), 4.58 (d, 1H, ²J 11.5, OCH₂Ph), 4.71 (s, 2H, OCH₂Ph), 4.77 (d, 1H, ²J 10.2, OCH₂Ph), 4.85 (d, 1H, ²J 10.2, OCH₂Ph), 4.95 (d, 1H, ²J 11.5, OCH₂Ph), 5.05 (s, 2H, OCH₂Ph), 5.32 (d, 1H, ³J7.6, NH), 5.32 (s, 1H, OH), 6.67 (d, 1H, ³J 7.4, NH), 6.93 (1s, 1H, NH), 7.21-7.38 (m, 25H, H_{Ar}).

¹³C NMR (CDCl₃, 75 MHz)

18.4 (CH₃); 21.9 (CH₂); 28.2 (CH₂); 31.2 (CH₂); 38.6 (CH₂N); 50.6 (CH); 52.1 (CH); 52.6 (OCH₃); 67.1 (CCH₂Ph); 68.6 (C6); 70.9 (C5); 73.3 and 73.5 (2OCH₂Ph); 74.1 (C4); 74.7 (OCH₂Ph); 74.8 (C2); 75.6 (OCH₂Ph); 80.7 (C3); 96.7 (t, 25.7 Hz, C1); 127.7; 127.8; 127.9; 128.0; 128.1; 128.2; 128.4 (2C); 128.6; 128.7 (Car.); 136.3; 137.9; 138.0; 138.4; 138.7 (quat. Car.); 164 (CF₂CONH); 172.6 (CONH); 172.7 (CO₂Me).

### Synthesis of Compound 21 (FIG. 15)

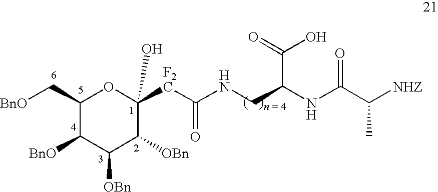

In a flask in an inert atmosphere containing compound 20 (575 mg; 0.586 mmol; 1 eq.) in solution in tetrahydrofuran (8 mL), an aqueous lithine solution (2M; 2 eq.) is added and the mixture is stirred overnight at ambient temperature. The medium is acidified with a 1M HCl solution (10 mL) and extracted with ethyl acetate (3×10 mL), and the organic phases are combined, washed with a saturated NaCl solution (20 mL) and concentrated directly. The acid 21 is isolated in this way as a pale yellow solid which may be used directly for the next step without further purifications with an unprocessed yield of 81%.

C₅₃H₅₉F₂N₃O₁₂ M=961 g.mol⁻¹

¹⁹F NMR (CDCl₃, 282 MHz)

−118.1 (1F, d, ²J_{F—F} 258.6), −119.9 (1F, d, ²J_{F—F} 258.6).

¹H NMR (CDCl₃, 300 MHz)

1.29 (tapp., 3H, ³J 9.7, CH₃), 1.10-1.41 (m, 4H, 2CH₂), 1.50-1.72 (m, 2H, CH₂), 2.88-2.95 (m, 1H, NCH₂), 3.22-3.32 (m, 1H, NCH₂), 3.49 (dapp, 6.0 Hz, 2H, H6), 3.89-3.92 (m, 2H, H3, H4), 4.13 (t, 6 Hz, 1H, H5), 4.22-4.43 (m, 3H, 2CH, H2); 4.38 (d, 2H, OCH₂Ph), 4.54 (d, 1H, ²J 11.5, OCH₂Ph), 4.67 (s, 2H, OCH₂Ph), 4.73 (d, 1H, ²J 10.2, OCH₂Ph), 4.81 (d, 1H, ²J 10.2, OCH₂Ph), 4.91 (d, 1H, ²J 11.4, OCH₂Ph), 4.97 (s, 2H, OCH₂Ph), 5.62 (d, 1H, ³J7.7, NH), 7.07 (1s, 2H, 2NH), 7.18-7.32 (m, 25H, H_{Ar}).

¹³C NMR (CDCl₃, 75 MHz)

19.0 (CH₃); 22.4 (CH₂); 28.8 (CH₂); 30.9 (CH₂); 39.0 (CH₂N); 50.9 (CH); 52.9 (CH); 67.6 (OCH₂Ph); 69.0 (C6); 71.2 (C5); 73.7 and 73.9 (2OCH₂Ph); 74.6 (C4); 75.1 (OCH₂Ph); 75.3 (C2); 75.9 (OCH₂Ph); 81.0 (C3); 97.1 (t, 27.4 Hz, C1); 128.1; 128.3; 128.5; 128.7; 128.8; 129.0; 129.1 (Car.); 136.7; 138.2; 138.4; 138.7; 138.9 (quat. Car.); 164.9 (CF₂CONH); 174.1 (CONH); 175.1 (CO₂H).

### Synthesis of Compound 22 (FIG. 16)

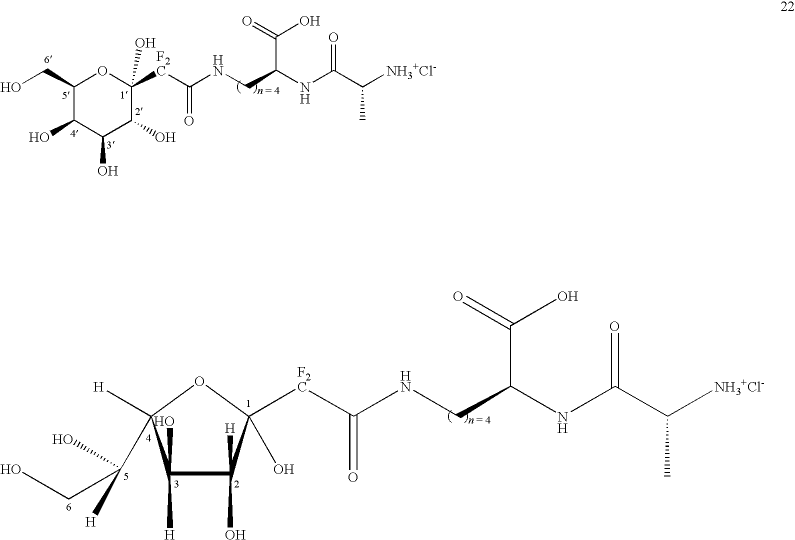

A flask containing the initial product 21 (458 mg; 0.473 mmol) in a mixture of tetrahydrofuran THF (15 mL) and 1N HCl (1.2 eq.) in the presence of a spatula tip of palladium on carbon Pd/C is placed in a hydrogen atmosphere. The mixture is left under stirring overnight and then filtered on a Millipore® filter. The mixture is then concentrated to obtain the product 16 in the form of a white solid with an 82% yield.

C₁₇H₃₀ClF₂N₃O₁₀ M=509 g.mol⁻¹

¹⁹F NMR (D₂O, 282 MHz)

−116.7 (1F, d, ²J_{F—F} 256.4), −117.8 (1F, d, ²J_{F—F} 256.4),

−118.2 (1F, d, ²J_{F—F} 256.4), −119.3 (1F, d, ²J_{F—F} 256.4).

¹H NMR (D₂O, 300 MHz)

1.51 (3H, d, ³J 7.1 Hz, CH₃), 1.25-1.90 (6H, m, 3CH₂), 3.25-3.32 (2H, m, NCH₂), 3.59-3.74 (m, 2H6′,2H6,H4,H5), 3.81-4.17 (m, H3′,CH,H4′,H5′,H2′), 4.32-4.41 (m, CH,H3,H2),

¹³C NMR (D₂O, 75 MHz)

18.4 (CH₃), 24.1 (CH₂), 29.5 (CH₂) 31.7 (CH₂), 41.1 (NCH₂), 50.8 (CH), 54.6 (CH), 62.7 (C6′); 64.2 (C6); 68.7 (C5′); 70.5 (C4′); 72.2 (C3′); 72.5 (C5); 74.1 (C2′); 75.6 (C3); 77.2 (C2); 81.9 (C4); 172.8 (CO).

### Synthesis of Compound 24 (FIG. 17)

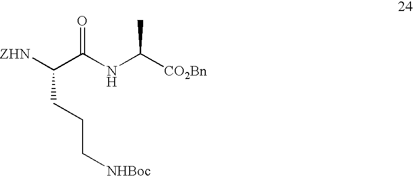

In a flask in an inert atmosphere containing Z-Orn(Boc)-OH 23 (1.00 g; 2.73 mmol; 1 eq) dissolved in dichloromethane (13 mL), carbonyldiimidazole (532 mg; 3.28 mmol; 1.2 eq) is added in small portions. The medium is stirred for one hour. To this product, a solution prepared in an inert atmosphere, consisting of Cl⁻⁺H₃N-Ala-OBn 7 (589 mg; 2.73 mmol; 1 eq) and DIEA (947 μL; 5.73 mmol; 2.1 eq) in dichloromethane (13 mL) is then added. This mixture is stirred for 12 hours. The medium is hydrolysed with a 1N hydrochloric acid solution, and the aqueous phase obtained is extracted three times with dichloromethane. The organic phases are pooled, dried on MgSO₄, filtered and concentrated. The unprocessed product is chromatographed on a silica column (Cyclohexane/Ethyl acetate 50/50 eluent) to obtain the compound 24 un the form of a white solid with a 70% yield.

C₂₈H₃₇N₃O₇ M=527.62 g.mol⁻¹

Rf: 0.44 (Cyclohexane/Ethyl acetate 50/50).

¹H NMR (CDCl₃, 300 MHz):

1.4 (m, 12H, C(CH₃)₃, CH₃); 1.47-1.53 (m, 2H, CH₂); 1.80-1.87 (m, 2H, CH₂); 3.01-3.33 (m, 1H, CH₂NH); 3.31-3.33 (m, 1H, CH₂NH); 4.4 (m, 1H, CH(Orn)); 4.55-4.62 (m, 1H, CH(Ala)); 4.73 (m, 1H, BocNH); 5.08 (s, 2H, OCH₂Ph); 5.08-5.11 (m, 1H, OCH₂Ph); 5.18 (d, 1H, OCH₂Ph ²J_{H—H}=12.2 Hz); 5.6 (m, H, ZNH); 7.0 (m, 1H, NH); 7.29-7.37 (m, 10H, Hₐᵣ)

¹³C NMR (CDCl₃, 75.5 MHz):

17.8 (CH₃); 26.3 (CH₂); 28.5 (C(CH₃)₃); 30.5 (CH₂); 39.2 (CH₂NH); 48.5 (CH(Ala)); 53.6 (CH(Orn)); 67.3 and 67.5 (2 OCH₂Ph); 79.4 (C(CH₃)₃; 128.2; 128.3 (2C); 128.5; 128.6; 128.7 (Car); 135.5; 136.4 (quat Car.); 156.4 and 156.8 (ZCO and BocCO); 171.8 and 172.5 (CONH and CO₂)

M(ES+): [M+H]⁺=528.87; [M+Na]⁺=551.00; [M+K]⁺=566.73; [2M+Na]⁺=1077.5 and [2M+K]⁺=1093.13

### Synthesis of Compound 25 (FIG. 18)

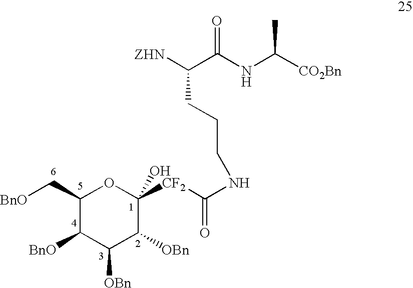

### Peptide Deprotection

In a flask in an inert atmosphere containing the peptide 24 (563 mg; 0.80 mmol; 1 eq) dissolved in dichloromethane (11 mL), trifluoroacetic acid (1.9 mL; 25.7 mmol; 20 eq) is added. The solution is stirred for 12 hours. The reaction medium is concentrated and co-evaporated 4 to 5 times with toluene. The residue is triturated with tert-butylmethylether and finally filtered and the deprotected peptide is obtained with a 72% yield.

### Coupling

In a flask in an inert atmosphere containing the acid 6 (508 mg; 0.80 mmol; 1 eq), the peptide deprotected above (433 mg; 0.80 mmol; 1 eq) in dimethylformamide (8 mL), HOBT (119 mg; 0.88 mmol; 1.1 eq) and NMM (220 μL; 2.00 mmol; 2.5 eq) are added. After 15 minutes, EDCI (169 mg; 0.88 mmol; 1.1 eq) is introduced. The solution is stirred for 48 hours.

The solvent is evaporated. The residue is taken up with dichloromethane and hydrolysed with a 1N hydrochloric acid solution. The aqueous phase is extracted three times with dichloromethane. The organic phases, once pooled are dried on MgSO₄, filtered and concentrated. The unprocessed product obtained in this way is chromatographed on a silica column to produce the compound 25 in the form of a white solid with a 27% yield.

C₅₉H₆₃F₂N₃O₁₂ M=1044.17 g.mol⁻¹

Rf=0.44 (Cyclohexane/Ethyl acetate 50/50 eluent)

¹⁹F NMR (CDCl₃), 282.5 MHz:

−117.7 (d, ²J_{F—F}=260 Hz); −120.3 (d, ²J_{F—F}=260 Hz)

¹H NMR (CDCl₃, 300 MHz):

1.33 (d, 3H, CH₃, ³J_{H—H}=7.3 Hz); 1.41-1.49 (m, 2H, CH₂); 1.73-1.81 (m, 2H, CH₂); 3.0 (m, 1H, CH₂NH); 3.47-3.64 (m, 3H, 2H₆ and CH₂NH); 3.93-3.96 (m, 2H, H₃ and H₄); 4.18 (t, 1H, H₅, ³J_{H5-H6}=6.5 Hz); 4.36 (d, 1H, H₂, ³J_{H2-H3}=9.5 Hz); 4.39-4.54 (m, 4H, CH(Orn), CH(Ala) and OCH₂Ph); 4.59 (d, 1H, OCH₂Ph, ²J_{H—H}=11.4 Hz); 4.75 (s, 2H, OCH₂Ph); 4.80 (d, 1H, OCH₂Ph, ²J_{H—H}=10.3 Hz); 4.89 (d, 1H, OCH₂Ph, ²J_{H—H}=10.3 Hz); 4.96 (d, 1H, OCH₂Ph, ²J_{H—H}=11.4 Hz); 5.07 (s, 2H, OCH₂Ph); 5.11 (s, 2H, OCH₂Ph); 5.2 (s, 1H, OH); 5.53 (d, 8.1 Hz, 1H, ZNH); 6.7 (d, 7.2 Hz, 1H, NH); 7.1 (m, 1H, NH); 7.27-7.34 (m, 30H, Hₐᵣ)

¹³C NMR (CDCl₃, 75.5 MHz):

17.6 (CH₃); 24.8 (CH₂); 30.0 (CH₂); 37.9 (CH₂N); 48.3 (CH(Ala)); 52.3 (CH(Orn)); 67.0 and 67.3 (2 OCH₂Ph); 68.4 (C6); 70.7 (C₅); 73.2 and 73.4 (2 OCH₂Ph); 74.0 (C₄); 74.7 (OCH₂Ph); 74.7 (C₂); 75.5 (OCH₂Ph); 80.7 (C₃); 96.7 (t, C₁ ¹J_{C—F}=27.6 Hz); 127.7; 127.8; 128.1 (2C); 128.2; 128.3; 128.4; 128.5; 128.6 (3C); 128.7 (Car); 135.4; 136.4; 137.7; 137.9; 138.3; 138.6 (quat C); 156.3 (ZNHCO); 164.3 (t, CF₂CO, ²J_{C—F}=28.2 Hz); 171.6 and 173.2 (CONH and CO₂)

M(ES+): [M+H]⁺=1044.33 and [M+Na]⁺=1066.47 and [M−H2O]⁺=1026.27

Elementary analysis: Theoretical % C 67.87% H 6.08% N 4.02
Experimental % C 67.96% H 6.39% N 3.46

### Synthesis of Compound 26 (FIG. 19)

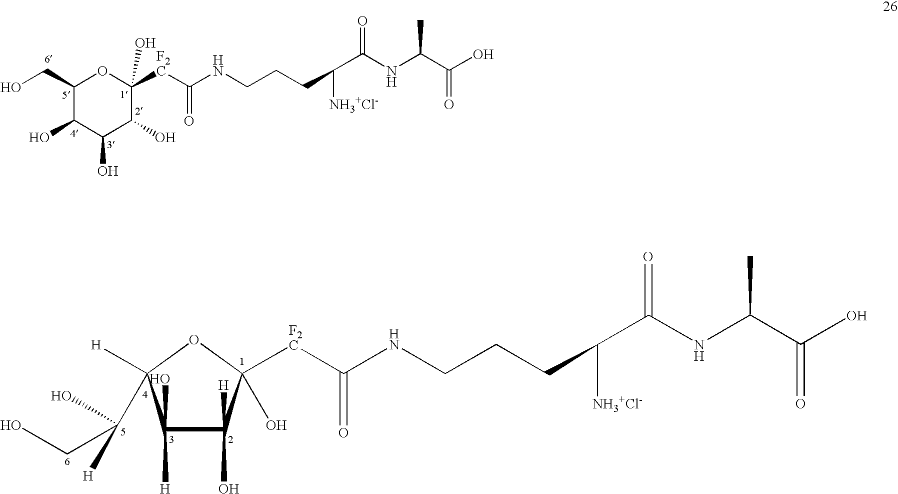

In a flask containing compound 25 (104 mg; 0.0996 mmol; 1 eq) dissolved in THF (10 mL), 1N hydrochloric acid (0.13 mL; 0.129 mmol; 1.3 eq), and a spatula tip of Pd/C are added. The solution is placed in a hydrogen atmosphere and stirred for 48 hours.

The medium is filtered on millipore and concentrated and compound 26 is obtained in the form of a pale yellow solid with a 97% yield.

C₁₆H₂₈ClF₂N₃O₁₀ M=495.86 g.mol⁻¹

¹⁹F NMR (D₂O, 282.5 MHz)

−119.4 (d, J_{F—F}=256 Hz); −120.6 (d, J_{F—F}=256 Hz); −121.0 (d, J_{F—F}=257 Hz); −122.1 (d, J_{F—F}=257 Hz).

¹H NMR (D₂O, 300 MHz)

1.43 (d; 6.7 Hz; CH₃); 1.66-1.78 (m; 2H; CH₂); 1.92 (m; 2H; CH₂); 3.34 (m; 2H; NHCH₂); 3.64-3.71 (m; 2H6′,2H6); 3.75-3.77 (m; H4 H5); 3.83-3.86 (m, H3′) 3.99-4.03 (m; CH(Orn),H4′,H5′,H2′); 4.16 (t, 7.5 Hz, H3); 4.4-4.43 (m; CH, H2).

¹³C NMR (D₂O, 75.5 MHz)

16.2 (CH₃); 20.8 (CH₂); 23.8 (CH₂); 39.0 (CH₂N); 49.1 (CH(Ala)); 53.2 (CH(Orn)); 61.1 (C6′); 62.5 (C6); 67.0 (C5′); 69.0 (C4′); 70.6 (C3′); 71.0 (C5); 72.5 (C2′); 74.0 (C3); 75.5 (C2); 80.2 (C4); 96.3 and 98.8 (C1 and C1′); 113.9 and 117.4 (CF₂); 169.6 and 176.3 (CO).

M(ES+): [M+H]⁺=461.2 and [M+Na]⁺=483.3 and [M−H₂O]⁺=441.3

### Synthesis of Compound 28 (FIG. 20)

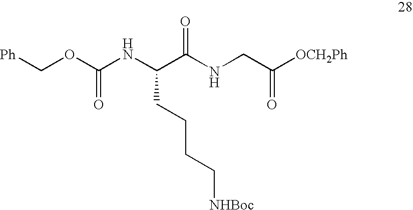

In a flask in an inert atmosphere containing Z-Lys(Boc)-OH 8 (1.90 g; 5.00 mmol; 1 eq) dissolved in dichloromethane (25 mL), carbonyldiimidazole (973 mg; 6.00 mmol; 1.2 eq) is added in small portions. The medium is stirred for one hour. To this product, a solution prepared in an inert atmosphere, consisting of Cl⁻⁺H₃N-Gly-OBn 27 (1.00 mg; 5.00 mmol; 1 eq) and DIEA (1.7 mL; 10.50 mmol; 2.1 eq) in dichloromethane (25 mL) is added. This mixture is stirred for 12 hours. The medium is hydrolysed with a 1N hydrochloric acid solution, and the aqueous phase obtained is extracted three times with dichloromethane. The organic phases are pooled, dried on MgSO₄, filtered, and concentrated.

The unprocessed product is chromatographed on a silica column (Cyclohexane/Ethyl acetate 50/50 eluent) to obtain compound 28 in the form of a white solid with an 89% yield.

C₂₈H₃₇N₃O₇ M=527.62 g.mol⁻¹

Rf: 0.41 (Ethyl acetate).

¹H NMR (CDCl₃, 300 MHz):

1.40 (s, 13H, C(CH₃)₃, 2CH₂); 1.67 (m, 1H, CH₂); 1.82 (m, 1H, CH₂); 3.07 (m, 2H, CH₂NH(Lys)); 4.04 (m, 2H, CH₂(Gly)); 4.21-4.23 (m, 1H, CH(Lys)); 4.71 (m, 1H, BocNH); 5.08 (s, 2H, OCH₂Ph); 5.16 (s, 2H, OCH₂Ph); 5.66 (d, 7.3 Hz, 1H, ZNH); 6.85 (m, 1H, NH); 7.30 (m, 10, Har).

¹³C NMR (CDCl₃, 75.5 MHz):

22.4 (CH₂); 28.5 (C(CH₃)₃); 29.7 (CH₂); 32.0 (CH₂); 39.8 (CH₂NH(Lys)); 41.1 (CH₂NH(Gly)); 54.8 (CH(Lys)); 67.2 and 67.3 (OCH₂Ph); 79.3 (C(CH₃)₃); 128.2; 128.3; 128.5; 128.6; 128.7 (3C) (Cₐᵣ); 135.2; 136.2 (C_{quat}); 156.4 (CO); 169.67 and 172.3 (CONH and CO₂)

M(ES+): [M+H]⁺=528.53 and [M+Na]⁺=550.53 and [M+K]⁺=566.27

Elementary analysis: Theoretical % C 63.74% H 7.07% N 7.96
Experimental % C 63.71% H 7.03% N 7.85

### Synthesis of Compound 29 (FIG. 21)

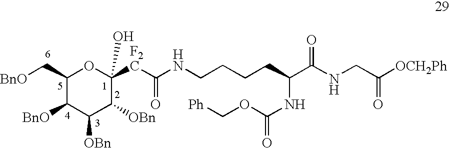

### Peptide Deprotection

In a flask in an inert atmosphere containing peptide 28 (2.04 g; 3.87 mmol; 1 eq) dissolved in dichloromethane (32 mL), trifluoroacetic acid (5.7 mL; 77.3 mmol; 20 eq) is added. The solution is stirred for 12 hours. The reaction medium is concentrated and co-evaporated 4 to 5 times with toluene to produce the deprotected peptide with a quantitative yield.

### Coupling

In a flask in an inert atmosphere containing the acid 6 (317 mg; 0.50 mmol; 1 eq), the peptide deprotected above (271 mg; 0.50 mmol; 1 eq) in dimethylformamide (5 mL), HOBT (74 mg; 0.55 mmol; 1.1 eq) and NMM (137 μL; 1.25 mmol; 2.5 eq) are added. After 15 minutes, EDCI (105 mg; 0.55 mmol; 1.1 eq) is introduced. The solution is stirred for 48 hours. The solvent is evaporated. The residue is taken up with dichloromethane and hydrolysed with a 1N hydrochloric acid solution. The aqueous phase is extracted three times with dichloromethane. The organic phases, once pooled, are dried on MgSO₄, filtered and concentrated. The unprocessed product obtained in this way is chromatographed on a silica column (Cyclohexane/Ethyl acetate 50/50 eluent) to produce compound 29 in the form of a white solid with a 47% yield.

C₅₉H₆₃F₂N₃O₁₂ M=1044.17 g.mol⁻¹

Rf=0.47 (Cyclohexane/Ethyl acetate 50/50 eluent)

¹⁹F NMR (CDCl₃), 282.5 MHz:

−116.6 (d, ²J_{F—F}=260 Hz); −122.1 (d, ²J_{F—F}=261 Hz)

¹H NMR (CDCl₃, 300 MHz):

1.30-1.81 (m, 6H, 3CH₂); 3.05-3.11 (m, 1H, CH₂NH(Lys)); 3.31-3.38 (m, 1H, CH₂NH(Lys)); 3.55 (d, 2H, H₆, ²J=6.2 Hz); 3.86-4.04 (m, 4H, H₃, H₄ and CH₂(Gly)); 4.16-4.20 (m, 2H, H₅ and CH(Lys)); 4.33 (d, 1H, H₂, ³J_{H2-H3}=10.0 Hz); 4.44 (d, 1H, OCH₂Ph, ²J_{H—H}=12.0 Hz); 4.49 (d, 1H, OCH₂Ph, ²J_{H—H}=12.0 Hz); 4.57 (d, 1H, OCH₂Ph, ²J_{H—H}=11.2 Hz); 4.74 (s, 2H, OCH₂Ph); 4.79 (d, 1H, OCH₂Ph, ²J_{H—H}=10.3 Hz); 4.87 (d, 1H, OCH₂Ph, ²J_{H—H}=10.3 Hz); 4.97 (d, 1H, OCH₂Ph, ²J_{H—H}=11.2 Hz); 5.12 (s, 2H, OCH₂Ph); 5.17 (s, 2H, OCH₂Ph); 5.53 (d, 1H, NHZ, ³J_{H—H}=7.4 Hz); 6.57 (m, 1H, NH(Gly)); 6.93 (m, 1H, NH); 7.28-7.35 (m, 30H, Hₐᵣ)

¹³C NMR (CDCl₃, 75.5 MHz):

21.9 (CH₂); 28.2 (CH₂); 31.8 (CH₂); 38.9 (CH₂NH(Lys)); 41.4 (CH₂(Gly)); 54.4 (CH(Lys)); 67.2 and 67.4 (2OCH₂Ph); 68.3 (C₆); 70.7 (C₅); 73.2 and 73.4 (OCH₂Ph); 74.1 (C₄); 74.6 (C₂); 74.8 and 75.5 (OCH₂Ph); 80.6 (C₃); 96.9 (t, C₁, ¹J_{C—F}=27 Hz); 127.6; 127.8 (2C); 128.0; 128.1; 128.2; 128.3 (3C); 128.4; 128.5; 128.6 (2C); 128.8 (Cₐᵣ); 135.2; 136.3; 137.9 (2C); 138.4; 138.6 (C_{quat}); 156.2 (ZNHCO); 163.9 (t, CF₂CO, ²J_{C—F}=27 Hz); 169.9 and 172.0 (NHCO and CO₂)

M(ES+): [M+H]⁺=1045.4 and [M+Na]⁺=1067.53.

Elementary analysis: Theoretical % C 67.87% H 6.08% N 4.02
Experimental % C 67.80% H 6.03% N 3.99

### Synthesis of Compound 30 (FIG. 22)

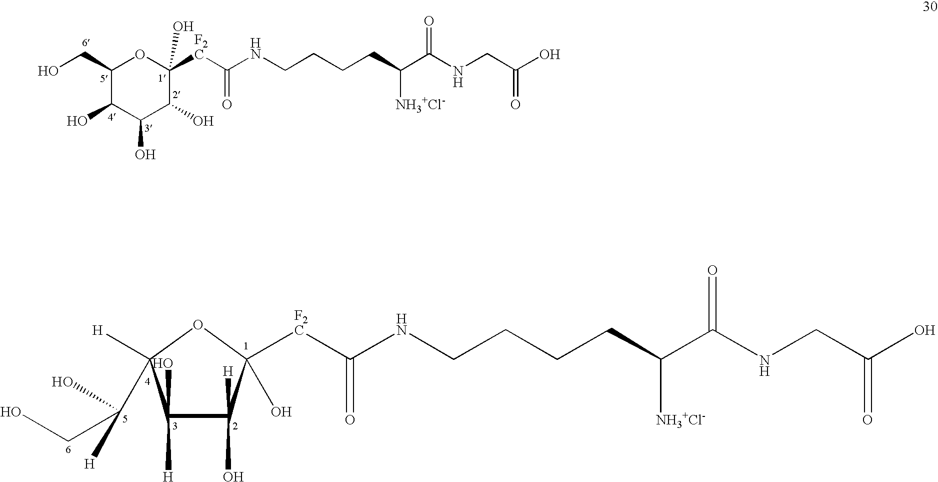

In a flask containing compound 29 (104 mg; 0.0996 mmol; 1 eq) dissolved in THF 3 mL, 1N hydrochloric acid (1.3 mL; 0.128 mmol; 1.3 eq), and a spatula tip of Pd/C are added. The solution is placed in a hydrogen atmosphere and stirred for 48 hours.

The medium is filtered on millipore and concentrated. Compound 30 is obtained in the form of a pale yellow solid with a 98% yield.

C₁₆H₂₈ClF₂N₃O₁₀ M=495.86 g.mol⁻¹

¹⁹F NMR (D₂O, 282.5 MHz)

−119.4 (d, J_{F—F}=256 Hz); −120.5 (d, J_{F—F}=256 Hz); −120.9 (d, J_{F—F}=257 Hz); −122.0 (d, J_{F—F}=257 Hz).

¹H NMR (D₂O, 300 MHz) 1.41-1.46 (m; 2H; CH₂); 1.53-1.60 (m; 2H; CH₂); 1.85-1.92 (m; 2H; CH₂); 3.27 (m; 2H; NHCH₂(Lys)); 3.57-3.74 (m; 2H6′,2H6;H4; H5); 3.83 (dd, 2.9 Hz and 9.3 Hz, H3′); 3.95-4.04 (m; CH₂,CH, H4′,H5′,H2′); 4.14 (t, 8.3 Hz, H3); 4.39 (d, 8 Hz, H2).

¹³C NMR (D₂O, 75.5 MHz)

21.6 (CH₂); 28.0 (CH₂); 30.6 (CH₂); 39.3 (CH₂N(Lys)); 41.4 (CH₂(Gly)); 53.4 (CH(Lys)); 61.1 (C6′); 62.6 (C6); 67.1 (C5′); 68.9 (C4′); 70.6 (C3′); 70.9 (C5); 72.5 (C2′); 73.9 (C3); 75.5 (C2); 80.2 (C4); 96.3 (t, 26 Hz) and 98.8 (t, 29 Hz) (C1 and C1′); 114.0 and 117.4 (CF₂); 163.9 (t, 28 Hz, CF₂CO); 170.7 and 173.2 (2CO).

M(ES+): [M+H]⁺=461.2 and [M+Na]⁺=483.3 and [M−H2O]⁺=441.3

### Synthesis of Compound 32 (FIG. 23)

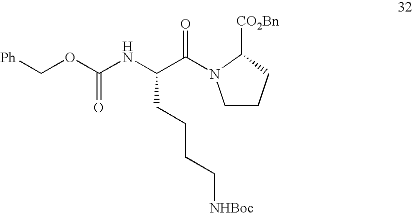

In a flask in an inert atmosphere containing Z-Lys(Boc)-OH 8 (760 mg; 2.00 mmol; 1 eq) dissolved in dichloromethane (20 mL), carbonyldiimidazole (389 mg; 2.40 mmol; 1.2 eq) is added in small portions. The medium is stirred for one hour. To this product, a solution prepared in an inert atmosphere, consisting of Cl⁻⁺HN-Pro-OBn 31 (483 mg; 2.00 mmol; 1 eq) and DIEA (690 μL; 4.20 mmol; 2.1 eq) in dichloromethane (20 mL) is added. This mixture is stirred for 12 hours. The medium is hydrolysed with a 1N hydrochloric acid solution, and the organic phase is extracted three times with dichloromethane. The organic phases are pooled, dried on MgSO₄, filtered and concentrated. The unprocessed product is chromatographed on a silica column (Cyclohexane/Ethyl acetate 50/50 eluent) to produce compound 32 in the form of a white solid with a 29% yield.

C₃₁H₄₁N₃O₇ M=567.69 g.mol⁻¹

Rf: 0.51 (Cyclohexane/Ethyl acetate 50/50).

¹H NMR (CDCl₃, 300 MHz):

1.3 (s, 9H, (CH₃)₃); 1.4-2.2 (m, 10H, 3 CH₂ Lys and 2 CH₂ Pro); 3.0 (m, 2H, CH₂NH(Lys)); 3.5 (m, 1H, CH₂N(Pro)); 3.6 (m, 1H, CH₂N(Pro)); 4.4 (m, 1H, CH(Lys)); 4.5 (m, 1H, CH(Pro)); 4.9 (d, 6.5 Hz, NHBoc), 4.97 (s, 2H, OCH₂Ph); 4.9 (m, 1H, OCH₂Ph); 5.09 (d, 1H, OCH₂Ph, ²J_{H—H}=12.3 Hz); 5.8 (d, 7.6 Hz, 1H, ZNH); 7.2 (m, 10H, Hₐᵣ)

¹³C NMR (CDCl₃, 75.5 MHz):

21.8 (CH₂); 24.7 (CH₂); 28.3 (C(CH₃)₃); 28.7 (CH₂); 29.2 (CH₂); 31.8 (CH₂); 39.8 (CH₂NH(Lys)); 46.8 (CH₂N(Pro)); 52.0 (CH(Lys)); 58.7 (CH(Pro)); 66.6 and 66.7 (OCH₂Ph); 78.6 (C(CH₃)₃; 127.8; 127.9; 128.0; 128.1; 128.3; 128.4 (Cₐᵣ); 135.4; 136.3 (C_{quat}); 155.9 and 156.0 (COZ and CO Boc); 170.7 (CONH); 171.6 (CO₂Bn)

M(ES+): [M+H]⁺=568.33; [M+Na]⁺=590.40; [M+K]⁺=560.27

### Synthesis of Compound 33 (FIG. 24)

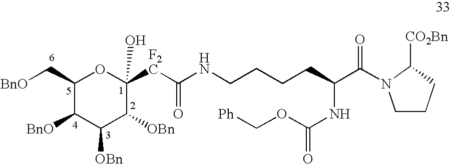

### Peptide Deprotection

In a flask in an inert atmosphere containing peptide 32 (318 mg; 0.56 mmol; 1 eq) dissolved in dichloromethane (6 mL), trifluoroacetic acid (833 μL; 11.22 mmol; 20 eq) is added. The solution is stirred for 12 hours. The reaction medium is concentrated and co-evaporated 4 to 5 times with toluene to quantitatively produce the deprotected peptide.

### Coupling

In a flask in an inert atmosphere containing the acid 6 (368 mg; 0.58 mmol; 1 eq), the peptide deprotected above (339 mg; 0.58 mmol; 1 eq) in dimethylformamide (6 mL), HOBT (87 mg; 0.64 mmol; 1.1 eq) and NMM (192 μL; 1.75 mmol; 3 eq) are added. After approximately 15 minutes, EDCI (123 mg; 0.64 mmol; 1.1 eq) is introduced. The solution is stirred for 48 hours. The solvent is evaporated. The residue is taken up with dichloromethane and hydrolysed with a 1N hydrochloric acid solution. The aqueous phase is extracted three times with dichloromethane. The organic phases, once pooled, are dried on MgSO₄, filtered and concentrated. The unprocessed product obtained in this way is chromatographed on a silica column (Cyclohexane/Ethyl acetate 50/50 eluent) to obtain compound 33 in the form of a white solid with a 29% yield.

C₆₂H₆₇F₂N₃O₁₂ M=1083.83 g.mol⁻¹

Rf=0.60 (Cyclohexane/Ethyl acetate 50/50 eluent)

¹⁹F NMR (CDCl₃; 282.5 MHz):

−116.1 (d, J_{F—F}=259 Hz); −122.9 (d, J_{F—F}=259 Hz)

¹H NMR (CDCl₃, 300 MHz):

1.2-1.9 (m, 10H, 3CH₂(Lys) and 2CH₂(Pro)); 3.1 (m, 1H, CH₂NH(Lys)); 3.3 (m, 1H, CH₂NH(Lys)); 3.5 (m, 4H, H₆ et CH₂N(Pro)); 4.0 (m, 2H, H₃ and H₄); 4.2 (t, 1H, H₅, ²J=6.4 Hz); 4.3 (d, 1H, H2, ²J_{H—H}=9.1); 4.4-4.5 (m, 1H, CH(Lys)); 4.44 (d, 1H, OCH₂Ph, ²J_{H—H}=12.1 Hz); 4.49 (d, 1H, OCH₂Ph, ²J_{H—H}=11.9 Hz); 4.5-4.6 (m, 1H, CH(Pro)); 4.55 (d, 1H, OCH₂Ph, ²J_{H—H}=11.3 Hz); 4.72 (s, 2H, OCH₂Ph); 4.76 (d, 1H, OCH₂Ph, ²J_{H—H}=10.4 Hz); 4.84 (d, 1H, OCH₂Ph, ²J_{H—H}=10.4 Hz); 4.93 (d, 1H, OCH₂Ph, ²J_{H—H}=11.3 Hz); 5.05 (d, 1H, OCH₂Ph, ²J_{H—H}=12.3 Hz); 5.07 (d, 1H, OCH₂Ph, ²J_{H—H}=9.3 Hz); 5.11 (d, 1H, OCH₂Ph, ²J_{H—H}=9.3 Hz); 5.17 (d, 1H, OCH²Ph, ²J_{H—H}=12.4 Hz); 5.4 (, 1H, OH); 5.6 (d, 8.3 Hz, 1H, ZNH); 7.1 (m, 1H, CONH); 7.3 (m, 30H, Hₐᵣ)

¹³C NMR (CDCl₃, 75.5 MHz):

21.6 (CH₂); 24.9 (CH₂); 27.9 (CH₂); 28.9 (CH₂); 32.1 (CH₂); 39.5 (CH₂NH(Lys)); 47.2 (CH₂N(Pro)); 52.1 (CH(Lys)); 59.0 (CH(Pro)); 66.9 and 67.2 (2OCH₂Ph); 68.2 (C₆); 70.7 (C₅); 73.2 and 73.5 (2OCH₂Ph); 74.3 (C₄); 74.6 (C₂); 74.9 and 75.4 (2OCH₂Ph); 80.5 (C3); 97.0 (t, C₁, ¹J_{C—F}=27 Hz); 127.9; 128.0; 128.3; 128.4; 128.5; 128.6; 128.7; 128.8; 128.9 (3C); 129.0 (Cₐᵣ); 135.5; 136.5; 138.0; 138.4; 138.8 (Car._{quat}); 156.0 (CONHZ)-164.0 (CF₂CO); 170.7 and 172.2 (CONH and CO₂)

M(ES+): [M+H]⁺=1084.6 and [M+Na]⁺=1106.67

M(ES−): [M−H]⁻=1082.27

Elementary analysis: Theoretical % C 68.68% H 6.23% N 3.88
Experimental % C 68.53% H 6.76% N 3.62

### Synthesis of Compound 34 (FIG. 25)

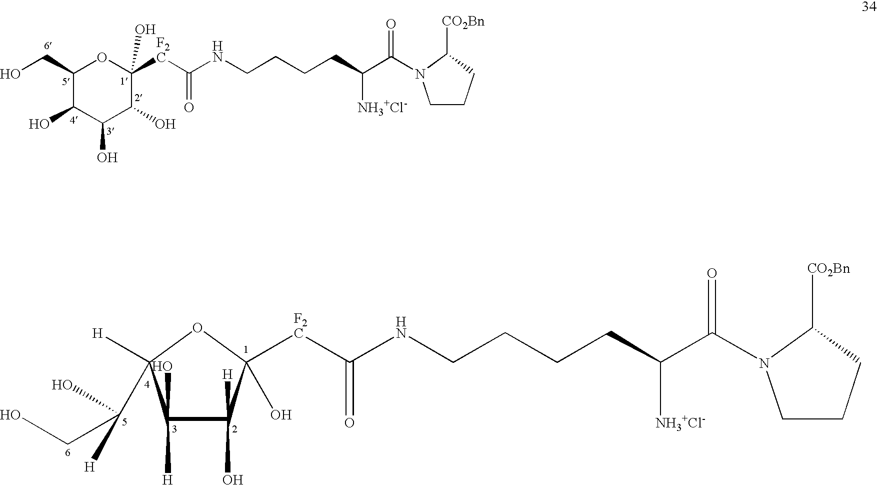

In a flask containing compound 33 (155 mg; 0.14 mmol; 1 eq) dissolved in THF (5 mL), 1N hydrochloric acid (175 μL; 0.19 mmol; 1.2 eq), and a spatula tip of Pd/C are added. The solution is placed in a hydrogen atmosphere and stirred for 48 hours. The medium is filtered on millipore and concentrated to obtain compound 34 in the form of a white solid with a quantitative yield.

C₁₉H₃₂ClF₂N₃O₁₀ M=536.01 g.mol⁻¹

¹⁹F NMR (D₂O, 282.5 MHz)

−119.4 (d, J_{F—F}=256 Hz); −120.5 (d, J_{F—F}=256 Hz); −121.0 (d, J_{F—F}=257 Hz); −122.0 (d, J_{F—F}=257 Hz).

¹H NMR (D₂O, 300 MHz)

1.45 (m; 2H; CH₂); 1.60 (m; 2H; CH₂); 1.89-1.92 (m; 2H; CH₂); 2.01 (m; 3H; CH₂); 2.33 (m; 1H; CH₂); 3.28-3.30 (m; 2H; NHCH₂(Lys)); 3.61-3.74 (m; 2H6′,2H6;H4; H5, NHCH₂(Pro), NCH′(Lys)); 3.83 (dd, 2.9 Hz and 9.9 Hz, H3′); 3.94-4.01 (m, H4′,H5′,H2′); 4.14 (t, 8.2 Hz, H3); 4.31 (t, 5.8 Hz, CH(Lys)); 4.39 (d, 8.2 Hz, H2); 4.38-4.51 (m, CH).

¹³C NMR (D₂O, 75.5 MHz)

21.3 (CH₂); 25.0 (CH₂); 28.1 (CH₂); 29.0 (CH₂); 29.7 (CH₂); 39.3 (CH₂N(Lys)); 48.0 (CH₂(Pro)); 52.0 (CH(Lys)); 53.4 (CH′(Lys)); 59.9 (C×H(Pro)); 61.1 (C6′); 62.6 (C6); 67.1 (C5′); 68.9 (C4′); 70.6 (C3′); 70.9 (C5); 72.5 (C2′); 73.9 (C3); 75.5 (C2); 80.2 (C4); 96.3 (t, 26 Hz) and 98.8 (t, 29 Hz) (C1 and C1′); 114 (t, 258 Hz, CF₂); 163.9 (2t, 28 Hz, CF₂CO); 168.8; 170.7 and 173.2 (CO).

### Synthesis of Compound 35 (FIG. 26)

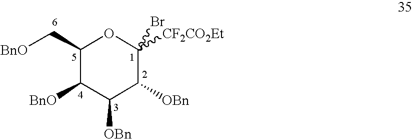

In a flask in an inert atmosphere containing difluoroester 5 (989 mg; 1.49 mmol: 1 eq.) in solution in anhydrous dichloromethane (7.5 mL) at −30° C., SOBr₂ (173 μL; 2,.24 mmol; 1.5 eq.) is added drop by drop. After 30 minutes, pyridine (181 μL; 2.24 mmol; 1.5 eq.) is introduced and the whole is left under stirring for a further 30 minutes at −30° C. A 2M HCl solution is added and the phase is extracted three times with dichloromethane. The organic phases are pooled, dried on MgSO₄, filtered and concentrated at reduced pressure. The unprocessed product 35 is obtained in the form of yellow crystals with a quantitative yield by weight without further purification.

C₃₈H₃₉BrF₂O₇ M=725.61 g.mol⁻¹

Rf: 0.54 (cyclohexane/ethyl acetate 8/2).

¹⁹F NMR (CDCl₃, 282.5 MHz)

−107.5 (d, 249 Hz, 1F); −111.9 (d, 249 Hz, 1F).

¹H NMR (CDCl₃, 300 MHz)

1.2 (t, 7.1 Hz, 3H, CH₃); 3.7 (dd, 5.7 and 9.4 Hz, 1H, 1H6); 3.8 (dd, 7.5 and 9.4 Hz, 1H, 1H6); 4.2 (m, 2H, H3 and H4); 4.2-4.3 (m, 2H, CH₂); 4.3 (m, 1H, H5); 4.5-5.1 (m, 9H, H2 and 4OCH₂Ph); 7.2 (m, 20H, Har)

¹³C NMR (CDCl₁₃, 75.5 MHz)

12.6 (CH₃); 62.4 (CH₂); 66.2 (C6); 72.0 (OCH₂Ph); 72.1 (C4); 72.4 (OCH₂Ph); 73.6 (OCH₂Ph); 73.7 (OCH₂Ph); 74.4 (C2); 74.5 (C5); 80.5 (C3); 102.4 (dd, 24 and 30 Hz, C1); 110.6 (tapp., 264 Hz, CF₂); 126.3; 126.4; 126.5; 126.6; 126.7; 126.8; 127.1; 127.2; 127.4 (Car.); 136.5; 136.9; 137.2; 137.3 (quat. Car.); 160.1 (tapp., 33 Hz, CO₂Et).

Mass (ESI+): 748.98 (M+Na); 774.88 (M+K).

### Synthesis of Compound 36 (FIG. 27)

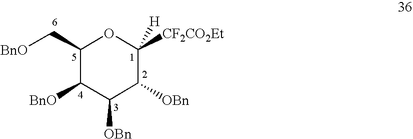

In a flask containing the ester 35 (519 mg; 0.72 mmol; 1 eq.) in solution in anhydrous toluene (15 mL) in a nitrogen atmosphere, previously distilled Bu₃SnH (291 μL; 1.08 mmol; 1.5 eq.) is added drop by drop. The whole is reflux-heated for 1 hour.

The product is concentrated and then purified on a silica column (cyclohexane/ethyl acetate 9.3/0.7 eluent) to retrieve the expected product 36 in the form of a colourless oil.

C₃₈H₄₀F₂O₇ M=646.7 g.mol⁻¹

¹⁹F NMR (CDCl₃, 282.5 MHz)

−116.7 (dd, 12 and 259 Hz, 1F); −118.2 (dd, 10 and 259 Hz, 1F).

¹H NMR (CDCl₃, 300 MHz)

1.05 (t, 7.2 Hz, 3H, CH₃); 3.47-3.53 (m, 3H, 2H6, H5); 3.57 (dd, 2.6 and 9.4 Hz, 1H, H3); 3.80-3.94 (m, 4H, H1, H4, CH₂); 4.09 (tapp., 9.5 Hz, 1H, H2); 4.31 (d, 11.8 Hz, 1H, OCH₂Ph); 4.37 (d, 11.8 Hz, 1H, OCH₂Ph); 4.51 (d, 11.5 Hz, 1H, OCH₂Ph); 4.54 (d, 10.4 Hz, 1H, OCH₂Ph); 4.56 (d, 11.7 Hz, 1H, OCH₂Ph); 4.65 (d, 11.7 Hz, 1H, OCH₂Ph); 4.85 (d, 11.7 Hz, 1H, OCH₂Ph); 4.86 (d, 10.4 Hz, 1H, OCH₂Ph); 7.20 (m, 20H, Har)

¹³C NMR (CDCl₃, 75.5 MHz)

14.2 (CH₃); 63.1 (CH₂); 68.9 (C6); 72.8 (OCH₂Ph); 73.6 (C4); 74.0 (OCH₂Ph); 74.1 (C2); 74.9 (OCH₂Ph); 75.3 (OCH₂Ph); 77.9 (tapp., 23 Hz, C1); 78.2 (C5); 84.7 (C3); 113.9 (tapp., 256 Hz, CF₂); 128.0-128.9 (Car.); 138.2; 138.3; 138.5; 139.0 (quat. Car.); 163.2 (tapp., 31 Hz, CO₂Et).

Mass (ESI+): 647.33 (M+H); 669.4 (M+Na).

### Synthesis of Compound 37 (FIG. 28)

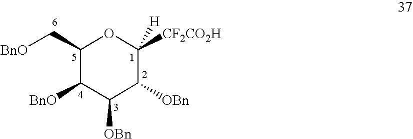

In a flask containing the ester 36 (0.4 g; 0.618 mmol, 1 eq.) in THF (5 mL), an aqueous lithine solution LiOH (30 mg; 1.25 mmol, 2.02 eq.) solubilised in a minimum quantity of water is added. The mixture is left for 12 hours under stirring and then taken up with ethyl acetate. The mixture is acidified with an aqueous 1N hydrochloric acid solution and extracted several times with ethyl acetate. The organic phases are pooled, dried on magnesium sulphate, filtered and concentrated.

The compound 37 is obtained in the form of a white oil with a quantitative yield.

C₃₆H₃₆F₂O₇ M=618.66 g.mol⁻¹

¹⁹F NMR (CDCl₃, 282.5 MHz)

−117.1 (d, J_{F—F}=260 Hz); −118.6 (d, J_{F—F}=260 Hz).

¹H NMR (CDCl₃, 300 MHz)

3.38-3.52 (m, 4H, 2H6, H5); 3.57 (dd, 2.6 and 9.4 Hz, 1H, H3); 3.79 (d, 2.4 Hz, 1H, H4); 3.79-3.88 (m, 1H, H1); 4.09 (t, 9.6 Hz, 1H, H2); 4.32 (d, 11.9 Hz, 1H, OCH₂Ph); 4.41 (d, 11.9 Hz, 1H, OCH₂Ph); 4.47-4.64 (m, 4H, 2OCH₂Ph); 4.81-4.86 (m, 2H, OCH₂Ph); 7.20 (m, 20H, Har)

### Synthesis of Compound 38 (FIG. 29)

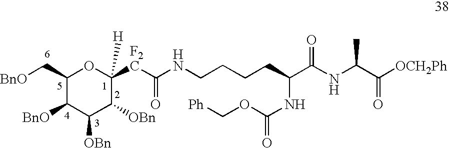

In a flask in an inert atmosphere containing the acid 37 (383 mg; 0.619 mmol; 1.0 eq.), the deprotected peptide (379 mg; 0.679 mmol; 1.1 eq.), 1-hydroxybenzotriazole HOBT (92 mg; 0.681 mmol; 1.1 eq.) and N-methylmorpholine NMM (204 μL; 1.8 mmol; 2.9 eq.) in DMF (5 mL), EDCI (130 mg; 0.678 mmol; 1.1 eq.) is added. The reaction is left under stirring for 24 hours, and the solvent is evaporated and the medium is taken up with dichloromethane. The medium is then extracted twice with 1M HCl (2*15 mL). The organic phase is collected, dried on magnesium sulphate, filtered and concentrated.

The mixture is then purified by means of chromatography on a silica column with a cyclohexane/ethyl acetate mixture as the eluent. After concentrating the collected fractions, the product 38 is presented in the form of a white solid with a 53% yield by weight.

C₆₀H₆₅F₂N₃O₁₁ M=1042.17 g.mol⁻¹

¹⁹F NMR (CDCl₃, 282.5 MHz)

−112. (dd, J 7.5 Hz and 260 Hz); −119.6 (d, J14.5 and 260 Hz).

¹H NMR (CDCl₃, 300 MHz)

1.17-1.19 (m, 2H, CH₂); 1.29-1.35 (m; 5H;CH₃; CH₂); 1.44-1.66 (m; 2H; CH₂); 2.90-2.94 (m; 1H; NHCH₂); 3.11-3.15 (m; 1H; NHCH₂); 3.43-3.54 (m; 4H; H6, H3;H5); 3.87 (d, 2.6 Hz, 1H, H4); 3.88-4.02 (m; 2H; CHNH(Lys); H1); 4.07 (t, 9.6 Hz, H2); 4.36 (s, 2H, OCH₂Ph); 4.46-4.52 (m; 3H; OCH₂Ph; CH (Ala)); 4.61-4.66 (m, 2H, OCH₂Ph); 4.77-4.87 (m, 2H, OCH₂Ph); 5.0 (s; 2H; OCH₂Ph); 5.01-5.13 (m, 2H, OCH₂Ph), 5.35 (d; 7.5 Hz, 1H; ZNH); 6.38 (d; 7.1; 1H; NH); 6.53 (1s, 1H; NH); 7.2 (m; 30H; Har.).

¹³C NMR (CDCl₃, 75.5 MHz)

18.4 (CH₃); 22.4 (CH₂); 28.8 (CH₂); 32.3 (CH₂); 39.2 (CH₂N); 48.6 (CH(Ala)); 54.9 (NCH(Lys)); 67.4 and 67.6 (2OCH₂Ph); 68.5 (C6); 73.1 (OCH₂Ph); 73.7 (C4); 73.8 (OCH₂Ph); 74.3 (C2); 75.1 (OCH₂Ph); 75.4 (OCH₂Ph); 77.1 (tapp, C1); 77.7 (C5); 84.4 (C3); 127.9-129 (Car.); 135.6; 136.6; 138.1; 138.5 (2C); 138.9 (quat. Car.); 156.0 (CONH(Z)); 171.6 (CONH); 172.9 (CO₂Bn).

### Synthesis of Compound 39 (FIG. 30)

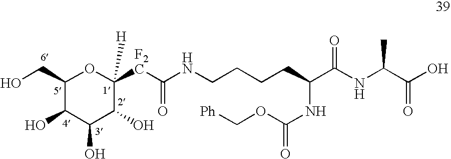

A flask containing the product 38 (400 mg; 0.384 mmol) in a mixture of tetrahydrofuran THF (5 mL) and 1N HCl (576 μL) and water (1.5 mL) in the presence of a spatula tip of palladium on carbon Pd/C is placed in a hydrogen atmosphere. The mixture is left under stirring overnight and filtered on a Millipore® filter. The mixture is then concentrated to obtain the product 39 in the form of a white solid with a quantitative yield.

C₁₇H₃₀ClF₂N₃O₉ M=493.88 g.mol⁻¹

¹⁹F NMR (D₂O, 282.5 MHz)

−116.7 (ddd, J=257 Hz, 17.2 Hz and 10.7 Hz,); −119.6 (ddd, J=257 Hz and 14 Hz).

¹H NMR (D₂O, 300 MHz)

1.32 (d; 7.3 Hz; CH₃); 1.31-1.34 (m, 2H, CH₂); 1.43-1.52 (m; 2H; CH₂); 1.75-1.83 (m; 2H; CH₂); 3.14-3.18 (m; 2H; NHCH₂); 3.42-3.61 (m; 4H, H6′, H5′, H3′); 3.72-3.79 (m; 2H H1′,H2′); 3.82-3.90 (m, 2H, CH(Lys), H4′); 4.24-4.32 (m; CH(Ala).

¹³C NMR (D₂O, 75.5 MHz)

16.3 (CH₃); 21.5 (CH₂); 27.9 (CH₂); 30.8 (CH₂); 39.4 (CH₂N); 49.1 (CH(Lys)); 53.2 (NCH(Ala)); 61.3 (C6′); 66.1 (C2′); 69.0 (C4′); 74.0 (C5′ or C3′); 77.3 (t, 24 Hz, C1); 79.6 (C3′ or C5′); 164.8 (d, 26 Hz, CO); 169.9 and 176.3 (2CO).

### Synthesis of Compound 41 (FIG. 31)

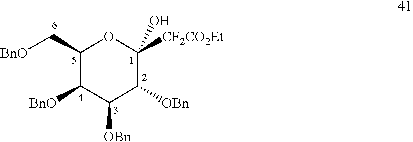

In a flask in an inert atmosphere containing previously activated zinc (3.34 g; 51 mmol; 7 eq.), THF (60 mL) is added and the mixed is thus reflux-heated. Lactone 40 (3.94 g; 7.3 mmol; 1 eq.) is slowly added with ethyl bromodifluoroacetate (2.83 mL; 22 mmol; 3 eq.) in THF (60 mL). The reaction is stirred at reflux for 3 hours. The mixture is cooled to ambient temperature and a 1M HCl solution (120 mL) is added. The mixture is filtered on a Buchner to remove the excess zinc. Dichloromethane (40 mL) is added to the solution. The two phases are separated and the aqueous phase is extracted a further two times with dichloromethane. The organic phases are dried on MgSO₄, filtered and concentrated at reduced pressure. The residue is purified on a chromatographic column on silica gel with a cyclohexane/ethyl acetate (8:2) mixture as the eluent in order to isolate the pure product 41 in the form of a colourless oil with a 75% yield.

C₃₈H₄₀F₂O₈ M=662.72 g.mol⁻¹

Rf=0.35, eluent: cyclohexane/ethyl acetate (8:2).

¹⁹F NMR (CDCl₃, 282 MHz)

−117.7 (d, 1F, ²J_{F—F}256.4), −120.1 (d, 1F, ²J_{F—F}256.4).

¹H NMR (CDCl₃, 300 MHz)

1.30 (t, 3H, ³J7.1, CH₃), 3.63-3.81 (m, 3H, H3, H6), 4.01-4.10 (m, 2H, H4, H5), 4.16 (s, 1H, H2), 4.29 (q, 2H, ³J 7.1, CH₂), 4.50-4.81 (m, 4H, 2OCH₂Ph), 4.85-4.92 (m, 4H, 2OCH₂Ph), 7.21-7.38 (m, 20H, H_{Ar}).

¹³C NMR (CDCl₃, 75 MHz)

14.3 (CH₃), 63.7 (CH₂), 68.6 (C6), 73.0 (C2), 73.8 (OCH₂Ph), 75.5 (OCH₂Ph), 75.7 (OCH₂Ph), 76.4 (OCH₂Ph), 77.8 (C3), 78.6 (C4), 83.7 (C5), 96.5 (t, ²J_{C—F} 25.5, C1), 128.0 (2C), 128.1 (2C), 128.1, 128.2, 128.3 (2C), 128.6, 128.7 (2C), 128.8 (2C), 128.8 (2C), 128.9 (2C) (Car.), 137.9, 138.3, 138.7, 138.7 (quat. Car.), 163.3 (t, ²J_{C—F} 30.3, CO).

### Synthesis of Compound 42 (FIG. 32)

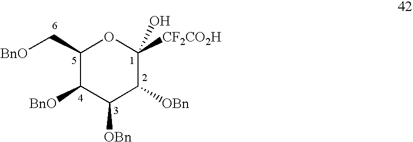

In a flask containing the ester 41 (615 mg; 0.93 mmol; 1 eq.) in THF (5 mL), an aqueous lithine solution LiOH (2M, 2 eq.) solubilised in a minimum quantity of water is added. The mixture is left for 12 hours under stirring and then taken up with ethyl acetate. The mixture is acidified with an aqueous 1N hydrochloric acid solution and then extracted several times with ethyl acetate. The organic phases are pooled, dried on magnesium sulphate, filtered and concentrated.

Compound 42 is obtained in the form of a white oil with a 90% yield.

C₃₆H₃₆F₂O₈ M=634.66 g.mol⁻¹

Rf=0.50; eluent: DCM/methanol (9:1).

¹⁹F NMR (CDCl₃, 282.5 MHz)

−117.2 (d, 1F, ²J_{F—F}259 Hz); −119.0 (d, 1F, ²J_{F—F}259 Hz).

¹H NMR (CDCl₃, 300 MHz)

3.4-3.5 (m, 2H, H3, 1H6); 3.5-3.6 (m, 1H, 1H6); 3.9 (m, 2H, H4, H5); 4.0 (m, 1H, H2); 4.4 (m, 8H; 4OCH₂Ph); 6.1 (s, 2H, OH, COOH); 7.0-7.3 (m, 20H, H_{Ar}).

¹³C NMR (CDCl₃, 75.5 MHz)

68.0 (C6); 71.4 (C2); 73.0; 74.9; 75.1; 75.9; 77.2 (C3); 77.9 (C4); 82.1 (C5); 94.9 (t, ²J_{CF} 27, C1); 126.6; 126.7; 126.9; 127.0; 127.0; 127.3; 127.3; 127.4; 127.5 (C ar.); 135.6; 136.2; 136.4; 137.1 (quat. Car.).

### Synthesis of Compound 43 (FIG. 33)

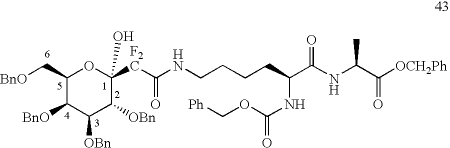

In a flask in an inert atmosphere containing the acid 42 (383 mg; 0.603 mmol; 1 eq.), the peptide deprotected above (370 mg; 0.664 mmol; 1.1 eq.), 1-hydroxybenzotriazole HOBT (0.090 g; 0.664 mmol; 1.1 eq.) and N-methylmorpholine NMM (0.198 mL; 1.8 mmol; 3 eq.) in DMF (10 mL), EDCI (127 mg; 0.664 mmol; 1.1 eq.) is added. The reaction is left under stirring for 24 hours, and the solvent is evaporated and the medium is taken up with dichloromethane. The medium is then extracted twice with 1M HCl (2*20 mL). The organic phase is collected, dried on magnesium sulphate, filtered and concentrated.

The mixture is then purified by means of chromatography on a silica column with a cyclohexane/ethyl acetate mixture as the eluent. After concentrating the collected fractions, the product 43 is presented in the form of a white solid with a 43% yield by weight.

C₆₀H₆₅F₂N₃O₁₂ M=1058.17 g.mol⁻¹

¹⁹F NMR (CDCl₃, 282.5 MHz)

−116.8 (d, J_{F—F}=260 Hz); −122.3 (d, J_{F—F}=259 Hz).

¹H NMR (CDCl₃, 300 MHz)

1.18-1.68 (m; 9H; CH₃; 3CH₂); 3.00-3.05 (m; 1H; NHCH₂); 3.21-3.26 (m; 1H; NHCH₂); 3.54-3.69 (m; 3H; H4, H6); 3.81 (d; 1H; 9.3 Hz, H2); 3.94 (t, 9.2 Hz, 2H, H5, H3); 4.0 (m; 1H; CHNH(Lys);); 4.34-4.50 (m; 4H; OCH₂Ph; CH (Ala)); 4.71-5.13 (m; 9H; OCH₂Ph); 5.2 (s; 1H, OH); 5.40 (d; 7.8; 1H; NH); 6.4 (m; 1H; NH); 6.9 (s; 1H; NH); 7.2 (m; 30H; Har.).

¹³C NMR (CDCl₃, 75.5 MHz)

18.1 (CH₃); 22.4 (CH₂); 28.4 (CH₂); 32, (CH₂); 39, (CH₂N); 48.7 (CH(Ala)); 54.8 (NCH Lys); 67.4 and 67.7 (2OCH₂Ph); 68.8 (C6); 72.0 (C5); 73.8 (OCH₂Ph); 75.4 (OCH₂Ph); 75.7 (OCH₂Ph); 76.4 (OCH₂Ph); 77.8 (C4); 78.7 (C2); 83.5 (C3); 128.2-129.0 (Car.); 135.6; 136.6; 138.0; 138.2; 138.4; 138.7 (quat. Car.); 156.5 (CO(Z)); 171.5 (CONH); 173.1 (CO₂Bn).

### Synthesis of Compound 44 (FIG. 34)

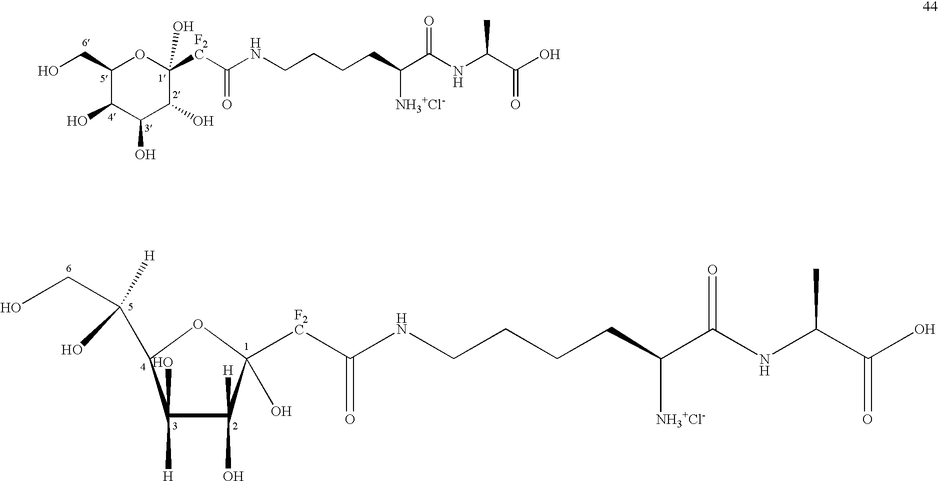

A flask containing the product 43 (220 mg; 0.208 mmol) in a mixture of tetrahydrofuran THF (3 mL), 1N HCl (312 μL) and water (approximately 1.7 mL) in the presence of a spatula tip of palladium on carbon Pd/C is placed in a hydrogen atmosphere. The mixture is left under stirring overnight and then filtered on a Millipore® filter. The mixture is then concentrated to obtain the product 44 in the form of a white solid with a 97% yield.

C₁₇H₃₀ClF₂N₃O₁₀ M=509.88 g.mol⁻¹

¹⁹F NMR (D₂O, 282.5 MHz)

−119.9 (s); −119.9 (s)

¹H NMR (D₂O, 300 MHz)

1.33 (m; 5H; CH₂ and CH₃); 1.48 (m; 2H; CH₂); 1.80 (m; 2H; CH₂); 3.17 (m; 2H; NHCH₂); 3.29-3.63 (m; 2H6, H4 H5,CH, H3; H2); 4.3 (m; CH).

### Cell Preservation Results

### Experiment 1

### Introduction:

The experiments were conducted in order to demonstrate the efficacy of compound 11 against exposure to UV.

### Results: (FIG. 35)

In the following experiments, the studies were conducted at 37° C. on adult primary skin fibroblasts. The cells were exposed to UV-C irradiation for 2 hours in the presence of AAGP derivatives.

Experimental protocols on Fibroblasts

| | |
|---|---|
| Temperatures used | 37° C. |
| Type de cellules | primary adult skin fibroblasts |
| Culture conditions | For these experiments, a serum-free |
| | medium with 0.5 mM EDTA was |
| | used. The cells were exposed to UV- |
| | C (254 nm) for 2 hours at ambient |
| | temperature, and then placed at 37° C. |
| | for the duration of the experiment. |
| Sampling times | At 0, 6, 18, 48, 72 days after UV |
| | exposure for AAGP-11 |
| AAGP compounds | AAGP-15 |
| AAGP concentration | 8.6 mM |

During these experiments, we observed that the compound AAGP-15 displays excellent protective properties.

Experimental Results

| Temperature | Preliminary Results | Comments |
|---|---|---|
| 37° C. | After 72 hours, all the | After 3 days, the compound |
| | control cells are dead, | AAGP-11 displays a very |
| | whereas with AAGP-11, | high level of cell |
| | 92% live cells are still | protection against UV. |
| | observed (FIG. 35). | After 4 days, 60% live |
| | | cells are still observed |
| | | with AAGP-11, whereas |
| | | almost all are dead in the |
| | | control (not shown in graph). |

### Experiment 2

### Introduction:

The purpose of the following experiment is to test the preservation activity of AAGP 11 compounds at low temperatures.

### Results: (FIG. 36)

The cells are thus incubated at −3° C. in the presence of the compound AAGP 11.

Experimental protocols on Fibroblasts

| | |
|---|---|
| Temperatures used | At −3° C. |
| Cell type | primary adult skin fibroblasts |
| Culture conditions | For these experiments, a serum-free medium |
| | with 0.5 mM EDTA was used. |
| | The cells were incubated at −3° C. |
| Sampling times | At 0, 1, 2, 4, and 6 days |
| AAGP compounds | AAGP-11 |
| AAGP concentration | 8.6 mM |

Similarly, in this experiment, a very high preservation activity of AAGP-11 on fibroblasts at low temperatures is observed.

Experimental Results

| Temperature | Preliminary results | Comments |
|---|---|---|
| −3° C. | After 6 days, the control cells | In these results, AAGP- |
| | are all dead whereas those | 11 displays a very high |
| | treated with AAGP-11 display | cell preservation activity. |
| | approximately 100% survival | |
| | (FIG. 36). | |

### Experiment 3

Studies were conducted on HELA cells, exposed to UVC, in the presence of the various synthesised compounds, at low concentrations.

Very satisfactory results are obtained for compound 11, but in view of FIGS. 37, 38 and 39, other derivatives improve the survival of these cells compared to the control. This is the case of the compounds: 39, 22, 30 and 34.

However, compounds 26, 12 and 44 give inferior results compared to the control.

1. The gem-difluorinated C-glycopeptide compound according to formula I: 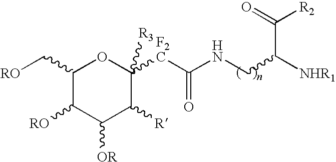
where n is an integer equal to 3 or 4,
R represents a hydrogen atom, a linear or branched alkyl, benzyl, acetyl, trimethylsilyl, tert-butyldimethylsilyl, tert-butyldiphenylsilyl group,
R′ represents OR, NR″R′″, N₃, or a phthalimide,
R″ and R′″, identical or different, represent a hydrogen atom or a linear or branched alkyl, aryl, benzyl, benzoyl, acetyl, alkyloxycarbonyl, allyloxycarbonyl, benzyloxycarbonyl group,
R₁ represents a hydrogen atom or a linear or branched alkyl, benzyl, alkylcarbamate, allylcarbamate, benzylcarbamate, acetyl group, R₁ may also represent an amino acid, but in this case R₂ only represents OR,
R₂ comprises an amino acid consisting of an alanine or a glycine, or a proline, but in this case R₁ represents a hydrogen atom or a linear or branched alkyl, benzyl, alkylcarbamate, allylcarbamate, benzylcarbamate, acetyl group, R₂ represents OR when R₁ represents an amino acid,
R₃ represents a hydrogen atom or a free or protected alcohol function,
and the derivatives thereof in the form of a physiologically or pharmaceutically acceptable base, addition salt to a mineral or organic acid, hydrate or solvate.

2. The compound according to claim 1, wherein the linear or branched alkyl groups are groups comprising 1 to 10 carbon atoms.

3. The medicinal product comprising as the active ingredient at least one gem-difluorinated C-glycopeptide according to formula I according to claim 1.

4. The formulation comprising at least one gem-difluorinated C-glycopeptide compound according to formula I according to claim 1.

5. The formulation according to claim 4, comprising a gem-difluorinated C-glycopeptide according to formula I alone or in a mixture and in any proportions.

6. The formulation according to claim 4, being presented in a pharmaceutical form rendering it suitable for a cosmetic or pharmaceutical or particularly dermatological use.

7. The formulation according to claim 4, being presented in a pharmaceutical form rendering it suitable to be ingested, injected or applied on the skin, lips, scalp and/or hair.

8. The formulation according to claim 4, comprising a physiologically or pharmaceutically acceptable medium and/or substrate.

9. The formulation according to claim 4, comprising other active ingredients.

10. The formulation suitable for topical application on the skin, lips, scalp and/or hair comprising a physiologically acceptable medium and/or substrate, comprising a gem-difluorinated C-glycopeptide compound according to formula I according to claim 1.

11. The cosmetic treatment method to protect the skin, lips and/or hair, scalp against oxidative stress and/or UV consisting of applying on the skin, lips and/or hair, scalp, a formulation comprising at least one physiologically acceptable medium and at least one gem-difluorinated C-glycopeptide compound according to formula I according to claim 1, in the form of a physiologically or pharmaceutically acceptable base, addition salt to a mineral or organic acid, hydrate or solvate.

12. A method for the preservation or cryopreservation of biological material, comprising containing the biological material with the gem-difluorinated C-glycopeptide compound of claim 1.

13. A method for the preservation of fibroblasts, comprising containing fibroblasts with the gem-difluorinated C-glycopeptide compound of claim 1.

14. A method to treat inflammation, comprising administering to a subject in need thereof a gem-difluorinated C-glycopeptide C-glycopeptide compound of claim 1.

15. (canceled)
